SEQUENCE LISTING (15 sequences)

SEQ ID NO: 1 - DNA, 1436 bp, Trichoderma reesei
atgaacaagtccgtggctccattgctgcttgcagcgtccatactatatggcggcgccgctgcacagcagactgtctggggccagtgtggaggtattggttggagcggacctacgaattgtgctcctggctcagcttgttcgaccctcaatccttattatgcgcaatgtattccgggagccactactatcaccacttcgacccggccaccatccggtccaaccaccaccaccagggctacctcaacaagctcatcaactccacccacgagctctggggtccgatttgccggcgttaacatcgcgggttttgactttggctgtaccacagagtgagtacccttgtttcctggtgttgctggctgaaaagttgggcgggtatacagcgatgcggactgcaagaacaccgccggtccgccaccatcaagatgtgggtggtaagcggcggtgttttgtacaactacctgacagctcactcaggaactgagaattaatggaagtcttgttacagtggcacttgcgttacctcgaaggtttatcctccgttgaagaacttcaccggctcaaacaactaccccgatggcatcggccagatgcagcacttcgtcaacgacgacgggatgactattttccgcttacctgtcggatggcagtacctcgtcaacaacaatttgggcggcaatcttgattccacgagcatttccaagtatgatcagcttgttcaggggtgcctgtctctgggcgcatactgcatcgtcgacatccacaattatgctcgatggaacggtgggatcattggtcagggcggccctactaatgctcaattcacgagcctttggtcgcagttggcatcaaagtacgcatctcagtcgagggtgtggttcggcatcatgaatgagccccacgacgtgaacatcaacacctgggctgccacggtccaagaggttgtaaccgcaatccgcaacgctggtgctacgtcgcaattcatctctttgcctggaaatgattggcaatctgctggggctttcatatccgatggcagtgcagccgccctgtctcaagtcacgaacccggatgggtcaacaacgaatctgatttttgacgtgcacaaatacttggactcagacaactccggtactcacgccgaatgtactacaaataacattgacggcgccttttctccgcttgccacttggctccgacagaacaatcgccaggctatcctgacagaaaccggtggtggcaacgttcagtcctgcatacaagacatgtgccagcaaatccaatatctcaaccagaactcagatgtctatcttggctatgttggttggggtgccggatcatttgatagcacgtatgtcctgacggaaacaccgactggcagtggtaactcatggacggacacatccttggtcagctcgtgtctcgcaagaaagtag

SEQ ID NO: 2 - protein, 397 aa, Trichoderma reesei
QQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNDDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 3 - protein, 418 aa, Trichoderma reesei
MNKSVAPLLLAASILYGGAAAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNDDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 4 - DNA, 31 bp, Artificial sequence
  Primer
gatcgtcgacatggtctccttcacctccctc

SEQ ID NO: 5 - DNA, 23 bp, Artificial sequence
  Primer
gcaacacctggcaattccttacc

SEQ ID NO: 6 - DNA, 24 bp, Artificial sequence
  Primer
tactatcaccacttcgacccggcc

SEQ ID NO: 7 - DNA, 17 bp, Artificial sequence
  Primer
tggctgtggagaagcgc

SEQ ID NO: 8 - DNA, 32 bp, Artificial sequence
  Primer
ggcacttgcgacacctcgaagatttatcctcc

SEQ ID NO: 9 - DNA, 28 bp, Artificial sequence
  Primer
cgaggtgtcgcaagtgccatctgtggta

SEQ ID NO: 10 - DNA, 34 bp, Artificial sequence
  Primer
gctacgtcgcacttcatctctttgcctggaaatg

SEQ ID NO: 11 - DNA, 26 bp, Artificial sequence
  Primer
gagatgaagtgcgacgtagcaccagc

SEQ ID NO: 12 - DNA, 29 bp, Artificial sequence
  Primer
cttggactcaaacaactccggtactcacg

SEQ ID NO: 13 - DNA, 32 bp, Artificial sequence
  Primer
ccggagttgtttgagtccaagtatttgtgcac

SEQ ID NO: 14 - DNA, 33 bp, Artificial sequence
  Primer
catgtgccgccaaatccaatatctcaaccagaa

SEQ ID NO: 15 - DNA, 32 bp, Artificial sequence
  Primer
attggatttggcggcacatgttttgtatgcag

(19) United States
(12) Patent Application Publication
(10) Pub. No.: US 2016/0326507 A1
(43) Pub. Date: Nov. 10, 2016

(54) VARIANTS OF GH FAMILY 5 ENDOGLUCANASE AND POLYNUCLEOTIDES ENCODING SAME

(71) Applicant: NOVOZYMES A/S, Bagsvaerd (DK)

(72) Inventors: James Lavigne, Wake Forest, NC (US); Daniel Sebastian Kolczynski, Ontario (CA); Brian R. Scott, West Sacramento, CA (US)

(73) Assignee: NOVOZYMES A/S, Bagsvaerd (DK)

(21) Appl. No.: 15/112,108

(22) PCT Filed: Jan. 22, 2015
(86) PCT No.: PCT/EP2015/051206

§ 371 Date: Jul. 15, 2016

(30) Foreign Application Priority Data
Jan. 23, 2014 (EP) 14152260.7

(51) Int. Cl. C12N 9/42

(52) U.S. Cl. CPC C12N 9/2437; C12Y 302/01004

(57) ABSTRACT

The present invention relates to GH Family 5 endoglucanase variants. The present invention also relates to polynucleotides encoding the variants; nucleic acid constructs, vectors, and host cells comprising the polynucleotides; and methods of using the variants.

### REFERENCE TO A SEQUENCE LISTING

This application contains a Sequence Listing in computer readable form, which is incorporated herein by reference.

### BACKGROUND OF THE INVENTION

1. Field of the Invention

The present invention relates to variants of GH Family 5 endoglucanases, polynucleotides encoding the variants, methods of producing the variants, and methods of using the variants in brewing.

2. Description of the Related Art

The conversion of cellulose polymers to simple sugars requires the concerted action of cellobiohydrolase (CBH) enzymes (EC 3.2.1.91 and EC 3.2.1.176) and endoglucanase (EG) enzymes (E.C. 3.2.1.4). Endoglucanases are cellulose-degrading enzymes that catalyze the hydrolysis of internal beta-1,4 glycosidic linkages in the cellulose polymer. Endoglucanase enzymes are produced by many bacteria and fungi and typically comprise a catalytic domain and a carbohydrate binding module (CBM) joined by a linker polypeptide known as a “linker” that serves as a flexible spacer between the CBM and the catalytic domain.

Endoglucanase enzymes are found in at least 13 distinct Glycosyl Hydrolase families based on their three-dimensional structures and catalytic mechanisms. GH Family 5 is one of the largest families of carbohydrate active enzymes and comprises thousands of enzymes from bacteria, eukaryotes and archae-bacteria, including both thermophilic and mesophilic organisms (Yennamalli et al., 2013, Biotechnol. Biofuels, 6:136). GH Family 5 enzymes include, but are not limited to endo-β-1,4-glucanase/cellulases (EC 3.2.1.4); chitosanases (EC 3.2.1.132); β-mannosidases (EC 3.2.1.25); glucan β-1,3-glucosidases (EC 3.2.1.58); licheninases (EC 3.2.1.73); glucan endo-1,6-β-glucosidases (EC 3.2.1.75); mannan endo-β-1,4-mannosidases (EC 3.2.1.78); endo-β-1,4-xylanases (EC 3.2.1.8); cellulose β-1,4-cellobiosidases (EC 3.2.1.91); β-1,3-mannanases (EC 3.2.1.-); and xyloglucan-specific endo-β-1,4-glucanases (EC 3.2.1.151).

While many GH 5 endoglucanases have been identified, only a few have been subject to protein engineering efforts to alter their biochemical or biophysical properties. GH Family endoglucanases that have been modified by rational design and/or random mutagenesis include Trichoderma reesei endoglucanase II (WO2011/109905; Qin et al., 2008, J. Biotechnol. 135:190-195; Wang et al., 2005, Biomol. Eng. 22:89-94), Acidothermus cellulolyticus E1 (Baker et al., 2005, Appl. Biochem. Biotechnol. 121-124:129-148), endoglucanase from Bacillus subtilis strain BME-15 (Lin et al., 2008, Appl. Microbiol. Biotechnol. 82:671-679; Park et al., 1993, Protein Eng. 6:921-926), and endoglucanase from Thermoanaerobacter tengcongensis Cel5A (Liang et al, J. Biotechnol. 154: 46-53).

Yet, despite these protein engineering efforts and the large diversity of sequences, there remains a need for GH5 endoglucanases with the high activity and stability at elevated temperatures associated with many industrial processes.

The present invention provides GH Family 5 endoglucanase variants with improved thermo activity and/or thermo stability compared to a parent GH Family 5 endoglucanase.

### SUMMARY OF THE INVENTION

The present invention relates to variants of a GH Family 5 endoglucanase, comprising amino acid substitutions at one or more (e.g., several) positions corresponding to positions 127, 171, 341, 336, 93, 97, 246, and 379 of SEQ ID NO: 2, wherein the variants have improved thermostability and/or thermoactivity.

The present invention also relates to polynucleotides encoding the variants; nucleic acid constructs, vectors, and host cells comprising the polynucleotides; and methods of producing the variants.

The present invention also relates to methods of using a GH Family 5 endoglucanase according to the invention in brewing; in particular to reduce the viscosity of a mash.

### BRIEF DESCRIPTION OF THE FIGURES

FIG. 1 shows vector maps of YEp352/PGKxylss-Cel5A (FIG. 1A) and YEp352/PGKxylss-Cel5A-G363A (FIG. 1B) used to express T. reesei EGII (TrEG2) and EGII-G363A (TrEG2(G363A)) from Saccharomyces cerevisiae and to perform random mutagenesis.

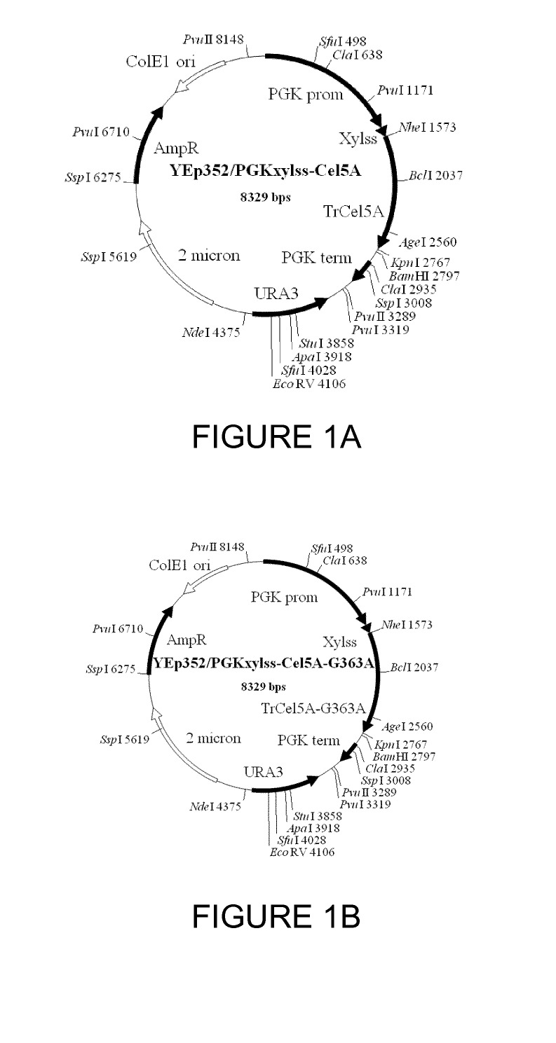

FIG. 2 shows thermoactivity data from one full round of screening. Plotted on the y-axis is TrEG2 activity for the high temperature (86° C.) and plotted on the x-axis is TrEG2 activity at the low temperature (70° C.) for both TrEG2 variants and parental control.

FIG. 3 shows the thermoactivity profiles for TrEG2 and TrEG2(T127N). Relative activity was calculated by dividing the activity at each temperature by the maximal activity.

FIG. 4 shows the thermoactivity profiles for TrEG2 and TrEG2(D341N). Relative activity was calculated by dividing the activity at each temperature by the maximal activity.

FIG. 5 shows the thermoactivity profiles for TrEG2 and TrEG2(Q336E). Relative activity was calculated by dividing the activity at each temperature by the maximal activity.

FIG. 6 shows the thermoactivity profiles for TrEG2 and TrEG2(V171I). Relative activity was calculated by dividing the activity at each temperature by the maximal activity.

FIG. 7 shows the thermoactivity profiles for TrEG2(G363A) and TrEG2(G363A-G379C). Relative activity was calculated by dividing the activity at each temperature by the maximal activity.

FIG. 8 shows the thermoactivity profiles for TrEG2-T127N-V171I-Q336E-D341N-G363A-G379C and TrEG-V93D-T127N-V171I-Q336E-D341N-G363A-G379C Relative activity was calculated by dividing the activity at each temperature by the maximal activity.

FIG. 9 shows the thermoactivity profiles for TrEG2-T127N-V171I-Q336E-D341N-G363A-G379C and TrEG-V97I-T127N-V171I-Q336E-D341N-G363A-G379C Relative activity was calculated by dividing the activity at each temperature by the maximal activity.

FIG. 10 shows the thermoactivity profiles for TrEG2-T127N-V171I-Q336E-D341N-G363A-G379C and TrEG-T127N-V171I-Q246H-Q336E-D341N-G363A-G379C Relative activity was calculated by dividing the activity at each temperature by the maximal activity.

FIG. 11 shows the thermoactivity profiles for TrEG2, TrEG2-V97I-T127N-V171I-Q246H-Q336E-D341N-G363A-G379C, and TrEG-V93D-V97I-T127N-V171I-Q246H-Q336E-D341N-G363A-G379C). Relative activity was calculated by dividing the activity at each temperature by the maximal activity.

FIG. 12 shows the thermo activity profiles for TrEG2, TrEG2(G363A), TrEG2-T127N-V171I-G363A-G379C and TrEG2-T127N-V171I-Q336E-D341N-G363A-G379C. Relative activity was calculated by dividing the activity at each temperature by the maximal activity.

### DEFINITIONS

Endoglucanase: The term “endoglucanase” means a carbohydrate-active enzyme classified under EC 3.2.1.4 that catalyzes the hydrolysis of (1→4)-β-D-glucosidic linkages in a cellulose polymer. There are several known assays that can be used for measuring endoglucanase activity. For example, endoglucanase activity can be monitored by measuring the enzyme-dependent creation of reducing sugars, which are quantified in subsequent chemical or chemi-enzymatic assays known to one of skill in the art. Examples 7 and 8 describe an endoglucanase assay which measures the release of reducing sugars from barley beta-glucan. Hydrolysis of polysaccharides can also be monitored by chromatographic methods that separate and quantify soluble mono-, di- and oligo-saccharides released by the enzyme. A further method involves determining the change in viscosity with time as the enzyme acts on the substrate. In addition, soluble colorimetric substrates may be incorporated into agar-medium on which a host microbe expressing and secreting a parent or variant GH Family 5 endoglucanase is grown. In such an agar-plate assay, activity of the endoglucanase is detected as a coloured or colourless halo around the individual microbial colony expressing and secreting an active cellulase. The specific activity of a GH Family 5 endoglucanase is determined by measuring the activity of the enzyme, typically in units of amount of glucose released per unit of time divided by the weight of the enzyme. For example, the specific activity may be determined in units of micromoles of glucose produced per minute per milligram of enzyme.

GH Family 5 Endoglucanase: As used herein, the term “Family 5 endoglucanase” or “Cel5” or “GH5 endoglucanase” encompasses an endoglucanase that contains a glycohydrolase (GH) Family 5 catalytic domain. Family 5 endoglucanases share a common (beta/alpha)₈-barrel fold and a catalytic mechanism resulting in a net retention of the anomeric sugar conformation. In Family 5 endoglucanases, both catalytic residues are glutamates (Cantarel et al., 2008, Nucleic. Acids. Res. 37:D233-D238). In T. reesei endoglucanase 11 (also referred to as EG2, TrEG2, EGII, Cel5A, or TrCel5A), residue E329 and E218 are the nucleophile and the acid/base respectively (Macarron et al., 1993, Biochem. J. 289: 87-873), and are highly conserved among family members (Wang et al., 1993, J. Biol. Chem. 268: 14096-14102). Other highly conserved amino acids among Family 5 endoglucanases are R130, H174, N217, H288, and Y290 residues. Many Family 5 cellulases, including T. reesei EG2, comprise a catalytic domain and a cellulose-binding domain joined by a flexible linker peptide (Stahlberg et al., 1988, Eur. J. Biochem. 173:179-183). In the case of T. reesei EG2, the N-terminal region (SEQ ID NO: 2 residues 1 to 36) is a cellulose binding domain (CBD) belonging to CBM (carbohydrate-binding module) Family 1 (Cantarel et al., 2008). The C-terminal domain (SEQ ID NO: 2 residues 71 to 397) is the glycohydrolase (GH) Family 5 catalytic domain which is responsible for the catalytic activity. The region between these two domains (SEQ ID NO: 2 residues 37 to 70) is a linker peptide rich in proline, serine, and threonine that serves as a flexible spacer between the CBD and the catalytic domain.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide that directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be RNA, messenger RNA or mRNA, genomic DNA, cDNA, synthetic DNA, or a combination thereof.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, transcriptional terminator, and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of a variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Fragment: The term “fragment” means a polypeptide having one or more (e.g., several) amino acids absent from the amino and/or carboxyl terminus of a mature polypeptide; wherein the fragment has endoglucanase activity. In one aspect, a fragment contains at least 361 amino acid residues (e.g., amino acids 37 to 397 of SEQ ID NO: 2), at least 327 amino acid residues (e.g., amino acids 71 to 391 of SEQ ID NO: 2), or at least 310 amino acid residues (e.g., amino acids 81 to 390 of SEQ ID NO: 2).

High stringency conditions: The term “high stringency conditions” means for probes of at least 100 nucleotides in length, prehybridization and hybridization at 42° C. in 5×SSPE, 0.3% SDS, 200 micrograms/mL sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Host cell: The term “host cell” means any cell type that is susceptible to transformation, transfection, transduction, or the like, with a nucleic acid construct or expression vector comprising a polynucleotide of the present invention. The term “host cell” encompasses any progeny of a parent cell that is not identical to the parent cell due to mutations that occur during replication.

Improved property: The term “improved property” means a characteristic associated with a variant that is improved compared to the parent. Such improved properties include, but are not limited to, thermal activity, thermo stability, stability under storage conditions, specific activity, substrate binding, substrate cleavage, substrate specificity, catalytic efficiency, catalytic rate, pH activity, pH stability, substrate stability, surface properties, chemical stability, and oxidation stability.

Isolated: The term “isolated” means a substance in a form or environment which does not occur in nature. Non-limiting examples of isolated substances include (1) any non-naturally occurring substance, (2) any substance including, but not limited to, any enzyme, enzyme variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance; enrichment of the substance within a composition). An isolated substance may be present in a fermentation broth sample.

Low stringency conditions: The term “low stringency conditions” means for probes of at least 100 nucleotides in length, prehybridization and hybridization at 42° C. in 5×SSPE, 0.3% SDS, 200 micrograms/mL sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 1 to 397 of SEQ ID NO: 2 or amino acids 22-418 of SEQ ID NO: 3, as determined by N-terminal sequencing of the purified EG2 protein (Saloheimo et al., 1988, Gene 63: 11-21). Analysis of the full-length amino acid sequence of SEQ ID NO: 3 using a program predicting signal peptides, e.g., SignalP (Nielsen et al., 1997, Protein Engineering 10: 1-6)] that predicts amino acids 1 to 21 of SEQ ID NO: 3 are a signal peptide. It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide having endoglucanase activity. In one aspect, the mature polypeptide coding sequence is nucleotides 64-329 and 509-1436 of SEQ ID NO: 1. SEQ ID NO: 1 contains one intron consisting of nucleotides 330-508. Experimental evidence (Saloheimo et al., 1988) has determined and the SignalP program (Nielsen et al., 1997) predicts that nucleotides 1 to 63 of SEQ ID NO: 1 encode a signal peptide.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

Mutant: The term “mutant” or “mutated” refers to a polynucleotide encoding a variant.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide, such that the control sequence directs expression of the coding sequence.

Parent or parental endoglucanase: The term “parent”, “parent endoglucanase”, or “parental endoglucanase” means an endoglucanase to which an alteration is made to produce the enzyme variants of the present invention. The parent may be a naturally occurring (wild-type) polypeptide or a variant or fragment thereof. Suitable parent endoglucanases include, but are not limited to, the fifteen known GH Family 5 endoglucanases shown in Table 1.

TABLE 1
Examples of known Family 5 cellulases

| GenPept | |
|---|---|
| Accession No. | Organism |
| AAA34213.1 | Trichoderma reesei |
| CAA61740.1 | Penicillium janthinellum |
| AAB03889.1 | Macrophomina phaseolina |
| AAC60541.1 | Cryptococcus flavus |
| EAA65878.1 | Aspergillus nidulans |
| BAB62317.1 | Aspergillus kawachii |
| AAB51451.1 | Macrophomina phaseolina |
| AAG59832.1 | Volvariella volvacea |
| AAL88714.2 | Thermoascus aurantiacus |
| AAC08587.1 | Aspergillus aculeatus |
| CAA53631.1 | Humicola insolens |
| AAB69347.1 | Orpinomyces joyonii |
| AAA75477.1 | Acidothermus cellulolyticus |
| ACI15227.1 | Bacillus subtilis |
| AAA22299.1 | Bacillus cellulosilyticus |

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence; wherein the subsequence encodes a fragment having enzyme activity. In one aspect, a subsequence contains at least 1083 nucleotides (e.g., nucleotides 172-329 and 509-1433 of SEQ ID NO: 1), at least 981 nucleotides (e.g., nucleotides 274-329 and 509-1415 of SEQ ID NO: 1), or at least 930 nucleotides (e.g., nucleotides 304-329 and 509-1412 SEQ ID NO: 1).

Variant: The term “variant” means a polypeptide having endoglucanase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. The variants of the present invention have at least 20%, e.g., at least 40%, at least 50%, at least 60%, at least 70%, at least 80%, at least 90%, at least 95%, or at least 100% of the endoglucanase activity of the mature polypeptide of SEQ ID NO: 2.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

Wild-type [enzyme]: The term “wild-type” endoglucanase means an endoglucanase expressed by a naturally occurring microorganism, such as a bacterium, yeast, or filamentous fungus found in nature.

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In one aspect, the cellulosic material is any biomass material. In another aspect, the cellulosic material is lignocellulose, which comprises cellulose, hemicelluloses, and lignin.

In an embodiment, the cellulosic material is agricultural residue, herbaceous material (including energy crops), municipal solid waste, pulp and paper mill residue, waste paper, or wood (including forestry residue).

In another embodiment, the cellulosic material is arundo, bagasse, bamboo, corn cob, corn fiber, corn stover, miscanthus, rice straw, sugar cane straw, switchgrass, or wheat straw.

In another embodiment, the cellulosic material is aspen, eucalyptus, fir, pine, poplar, spruce, or willow.

In another embodiment, the cellulosic material is algal cellulose, bacterial cellulose, cotton linter, filter paper, microcrystalline cellulose (e.g., AVICEL®), or phosphoric-acid treated cellulose.

In another embodiment, the cellulosic material is an aquatic biomass. As used herein the term “aquatic biomass” means biomass produced in an aquatic environment by a photosynthesis process. The aquatic biomass can be algae, emergent plants, floating-leaf plants, or submerged plants.

The cellulosic material may be used as it is or it may be subjected to pretreatment, using conventional methods known in the art. In a preferred aspect, the cellulosic material is pretreated.

Hemicellulosic material: The term “hemicellulosic material” means any material comprising hemicelluloses. Hemicelluloses include xylan, glucuronoxylan, arabinoxylan, glucomannan, and xyloglucan. These polysaccharides contain many different sugar monomers. Sugar monomers in hemicellulose can include xylose, mannose, galactose, rhamnose, and arabinose. Hemicelluloses contain most of the D-pentose sugars. Xylose is in most cases the sugar monomer present in the largest amount, although in softwoods mannose can be the most abundant sugar. Xylan contains a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67. Hemicellulosic material is also known herein as “xylan-containing material”.

Sources for hemicellulosic material are essentially the same as those for cellulosic material described herein.

In the processes of the present invention, any material containing hemicellulose may be used. In a preferred aspect, the hemicellulosic material is lignocellulose.

### Conventions for Designation of Variants

For purposes of the present invention, the mature polypeptide disclosed in SEQ ID NO: 2 (T. reesei EG2 or TrEG2) is used to determine the corresponding amino acid residue in another GH Family 5 endoglucanase. The amino acid sequence of another GH Family 5 endoglucanase is aligned with the mature polypeptide disclosed in SEQ ID NO: 2, and based on the alignment, the amino acid position number corresponding to any amino acid residue in the mature polypeptide disclosed in SEQ ID NO: 2 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another GH Family 5 endoglucanase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537:_39-64; Katoh and Toh, 2010, Bioinformatics 26:_1899-1900), and EMBOSS EMMA employing ClustalW (1.83 or later; Thompson et al., 1994, Nucleic Acids Research 22: 4673-4680), using their respective default parameters.

When the other GH Family 5 endoglucanase has diverged from the mature polypeptide of SEQ ID NO: 2 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions.

For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions.

For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions.

For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple Amino Acid Substitutions.

Variants comprising multiple amino acid substitutions are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different Amino Acid Substitutions.

Where different amino acid substitutions can be introduced at a position, the different amino acid substitutions are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The present invention relates to variants of GH Family 5 endoglucanases, comprising a substitution at one or more (e.g., several) positions corresponding to positions 127, 171, 341, 336, 93, 97, 246, and 379 of the mature polypeptide of SEQ ID NO: 2, wherein the variant has endoglucanase activity.

### Variants

The present invention provides variants of GH Family 5 endoglucanases, comprising a substitution at one or more (e.g., several) positions corresponding to positions 127, 171, 341, 336, 93, 97, 246, and 379 of SEQ ID NO: 2, wherein the variant has endoglucanase activity.

In an embodiment, the variant has sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent endoglucanase.

In another embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the mature polypeptide of SEQ ID NO: 2.

In one aspect, the number of amino acid substitutions in the variants of the present invention is 1-20, e.g., 1-10 or 1-5, such as 1, 2, 3, 4, 5, 6, 7, 8, 9, 10, 11, 12, 13, 14, 15, 16, 17, 18, 19, or 20 amino acid substitutions.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermo stability and/or thermal activity of the polypeptide, change the pH optimum, and the like. For example, the variants may comprise a Val to Asp substitution at position 93 of SEQ ID NO: 2, a Thr to Asn substitution at position 127 of SEQ ID NO: 2, a Gln to His substitution at position 246 of SEQ ID NO: 2, a Gln to Glu substitution at position 336 of SEQ ID NO: 2, and/or a Gly to Cys substitution at position 379 of SEQ ID NO: 2.

In another aspect, a variant comprises an amino acid substitution at one or more (e.g., several) positions corresponding to positions 127, 171, 341, 336, 93, 97, 246, and 379 of SEQ ID NO: 2. In another aspect, a variant comprises an amino acid substitution at two positions corresponding to any of positions 127, 171, 341, 336, 93, 97, 246, and 379 of SEQ ID NO: 2. In another aspect, a variant comprises an amino acid substitution at three positions corresponding to any of positions 127, 171, 341, 336, 93, 97, 246, and 379 of SEQ ID NO: 2. In another aspect, a variant comprises an amino acid substitution at four positions corresponding to any of positions 127, 171, 341, 336, 93, 97, 246, and 379 of SEQ ID NO: 2. In another aspect, a variant comprises an amino acid substitution at five positions corresponding to any of positions 127, 171, 341, 336, 93, 97, 246, and 379 of SEQ ID NO: 2. In another aspect, a variant comprises an amino acid substitution at six positions corresponding to any of positions 127, 171, 341, 336, 93, 97, 246, and 379 of SEQ ID NO: 2. In another aspect, a variant comprises an amino acid substitution at seven positions corresponding to any of positions 127, 171, 341, 336, 93, 97, 246, and 379 of SEQ ID NO: 2. In another aspect, a variant comprises an amino acid substitution at each position corresponding to positions 127, 171, 341, 336, 93, 97, 246, and 379 of SEQ ID NO: 2.

In another aspect, the variant comprises or consists of an amino acid substitution at a position corresponding to position 127 of SEQ ID NO: 2. In another aspect, the amino acid at a position corresponding to position 127 is substituted with Ala, Arg, Asn, Asp, Cys, Gln, Glu, Gly, His, Ile, Leu, Lys, Met, Phe, Pro, Ser, Trp, Tyr, or Val, preferably with Asn. In another aspect, the variant comprises or consists of the substitution T127N of the mature polypeptide of SEQ ID NO: 2.

In another aspect, the variant comprises or consists of an amino acid substitution at a position corresponding to position 171 of SEQ ID NO: 2. In another aspect, the amino acid at a position corresponding to position 171 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile. In another aspect, the variant comprises or consists of the substitution V171I of the mature polypeptide of SEQ ID NO: 2.

In another aspect, the variant comprises or consists of an amino acid substitution at a position corresponding to position 341 of SEQ ID NO: 2. In another aspect, the amino acid at a position corresponding to position 341 is substituted with Ala, Arg, Asn, Asp, Cys, Gin, Glu, Gly, His, Ile, Leu, Lys, Met, Phe, Pro, Ser, Thr, Trp, Tyr, or Val, preferably with Asn. In another aspect, the variant comprises or consists of the substitution D341N of the mature polypeptide of SEQ ID NO: 2.

In another aspect, the variant comprises or consists of an amino acid substitution at a position corresponding to position 336 of SEQ ID NO: 2. In another aspect, the amino acid at a position corresponding to position 336 is substituted with Ala, Arg, Asn, Asp, Cys, Gin, Glu, Gly, His, Ile, Leu, Lys, Met, Phe, Pro, Ser, Thr, Trp, Tyr, or Val, preferably with Glu. In another aspect, the variant comprises or consists of the substitution Q336E of the mature polypeptide of SEQ ID NO: 2.

In another aspect, the variant comprises or consists of an amino acid substitution at a position corresponding to position 93 of SEQ ID NO: 2. In another aspect, the amino acid at a position corresponding to position 93 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another aspect, the variant comprises or consists of the substitution V93D of the mature polypeptide of SEQ ID NO: 2.

In another aspect, the variant comprises or consists of an amino acid substitution at a position corresponding to position 97 of SEQ ID NO: 2. In another aspect, the amino acid at a position corresponding to position 97 is substituted with Ala, Arg, Asn, Asp, Cys, Gin, Glu, Gly, His, Ile, Leu, Lys, Met, Phe, Pro, Ser, Thr, Trp, Tyr, or Val, preferably with Ile. In another aspect, the variant comprises or consists of the substitution V91I of the mature polypeptide of SEQ ID NO: 2.

In another aspect, the variant comprises or consists of an amino acid substitution at a position corresponding to position 246 of SEQ ID NO: 2. In another aspect, the amino acid at a position corresponding to position 246 is substituted with Ala, Arg, Asn, Asp, Cys, Gin, Glu, Gly, His, Ile, Leu, Lys, Met, Phe, Pro, Ser, Thr, Trp, Tyr, or Val, preferably with His. In another aspect, the variant comprises or consists of the substitution Q246H of the mature polypeptide of SEQ ID NO: 2.

In another aspect, the variant comprises or consists of an amino acid substitution at a position corresponding to position 379 of SEQ ID NO: 2. In another aspect, the amino acid at a position corresponding to position 379 is substituted with Ala, Arg, Asn, Asp, Cys, Gin, Glu, Gly, His, Ile, Leu, Lys, Met, Phe, Pro, Ser, Thr, Trp, Tyr, or Val, preferably with Cys. In another aspect, the variant comprises or consists of the substitution G379C of the mature polypeptide of SEQ ID NO: 2.

In another aspect, the variant comprises or consists of an alteration at positions corresponding to positions 127 and 171 of SEQ ID NO: 2, such as those described above.

In another aspect, the variant comprises or consists of amino acid substitutions at positions corresponding to positions 127 and 341 of SEQ ID NO: 2, such as those described above.

In another aspect, the variant comprises or consists of amino acid substitutions at positions corresponding to positions 127 and 336 of SEQ ID NO: 2, such as those described above.

In another aspect, the variant comprises or consists of amino acid substitutions at positions corresponding to positions 127 and 93 of SEQ ID NO: 2, such as those described above.

In another aspect, the variant comprises or consists of amino acid substitutions at positions corresponding to positions 127 and 97 of SEQ ID NO: 2, such as those described above.

In another aspect, the variant comprises or consists of amino acid substitutions at positions corresponding to positions 127 and 246 of SEQ ID NO: 2, such as those described above.

In another aspect, the variant comprises or consists of amino acid substitutions at positions corresponding to positions 127 and 379 of SEQ ID NO: 2, such as those described above.

In another aspect, the variant comprises or consists of amino acid substitutions at positions corresponding to positions 171 and 341 of SEQ ID NO: 2, such as those described above.

In another aspect, the variant comprises or consists of amino acid substitutions at positions corresponding to positions 171 and 336 of SEQ ID NO: 2, such as those described above.

In another aspect, the variant comprises or consists of amino acid substitutions at positions corresponding to positions 171 and 93 of SEQ ID NO: 2, such as those described above.

In another aspect, the variant comprises or consists of amino acid substitutions at positions corresponding to positions 171 and 97 of SEQ ID NO: 2, such as those described above.

In another aspect, the variant comprises or consists of amino acid substitutions at positions corresponding to positions 171 and 246 of SEQ ID NO: 2, such as those described above.

In another aspect, the variant comprises or consists of amino acid substitutions at positions corresponding to positions 171 and 379 of SEQ ID NO: 2, such as those described above.

In another aspect, the variant comprises or consists of amino acid substitutions at positions corresponding to positions 341 and 336 of SEQ ID NO: 2, such as those described above.

In another aspect, the variant comprises or consists of amino acid substitutions at positions corresponding to positions 341 and 93 of SEQ ID NO: 2, such as those described above.

In another aspect, the variant comprises or consists of amino acid substitutions at positions corresponding to positions 341 and 97 of SEQ ID NO: 2, such as those described above.

In another aspect, the variant comprises or consists of amino acid substitutions at positions corresponding to positions 341 and 246 of SEQ ID NO: 2, such as those described above.

In another aspect, the variant comprises or consists of amino acid substitutions at positions corresponding to positions 341 and 379 of SEQ ID NO: 2, such as those described above.

In another aspect, the variant comprises or consists of amino acid substitutions at positions corresponding to positions 336 and 93 of SEQ ID NO: 2, such as those described above.

In another aspect, the variant comprises or consists of amino acid substitutions at positions corresponding to positions 336 and 97 of SEQ ID NO: 2, such as those described above.

In another aspect, the variant comprises or consists of amino acid substitutions at positions corresponding to positions 336 and 246 of SEQ ID NO: 2, such as those described above.

In another aspect, the variant comprises or consists of amino acid substitutions at positions corresponding to positions 336 and 379 of SEQ ID NO: 2, such as those described above.

In another aspect, the variant comprises or consists of amino acid substitutions at positions corresponding to positions 93 and 97 of SEQ ID NO: 2, such as those described above.

In another aspect, the variant comprises or consists of amino acid substitutions at positions corresponding to positions 93 and 246 of SEQ ID NO: 2, such as those described above.

In another aspect, the variant comprises or consists of amino acid substitutions at positions corresponding to positions 93 and 379 of SEQ ID NO: 2, such as those described above.

In another aspect, the variant comprises or consists of amino acid substitutions at positions corresponding to positions 97 and 246 of SEQ ID NO: 2, such as those described above.

In another aspect, the variant comprises or consists of amino acid substitutions at positions corresponding to positions 97 and 379 of SEQ ID NO: 2, such as those described above.

In another aspect, the variant comprises or consists of amino acid substitutions at positions corresponding to positions 246 and 379 of SEQ ID NO: 2, such as those described above.

In another aspect, the variant comprises or consists of amino acid substitutions at positions corresponding to positions 127, 171, and 341 of SEQ ID NO: 2, such as those described above.

In another aspect, the variant comprises or consists of amino acid substitutions at positions corresponding to positions 127, 171, and 336 of SEQ ID NO: 2, such as those described above.

In another aspect, the variant comprises or consists of amino acid substitutions at positions corresponding to positions 127, 171, and 93 of SEQ ID NO: 2, such as those described above.

In another aspect, the variant comprises or consists of amino acid substitutions at positions corresponding to positions 127, 171, and 97 of SEQ ID NO: 2, such as those described above.

In another aspect, the variant comprises or consists of amino acid substitutions at positions corresponding to positions 127, 171, and 246 of SEQ ID NO: 2, such as those described above.

In another aspect, the variant comprises or consists of amino acid substitutions at positions corresponding to positions 127, 171, and 379 of SEQ ID NO: 2, such as those described above.

In another aspect, the variant comprises or consists of amino acid substitutions at positions corresponding to positions 127, 341, and 336 of SEQ ID NO: 2, such as those described above.

In another aspect, the variant comprises or consists of amino acid substitutions at positions corresponding to positions 127, 341, and 93 of SEQ ID NO: 2, such as those described above.

In another aspect, the variant comprises or consists of amino acid substitutions at positions corresponding to positions 127, 341, and 97 of SEQ ID NO: 2, such as those described above.

In another aspect, the variant comprises or consists of amino acid substitutions at positions corresponding to positions 127, 341, and 246 of SEQ ID NO: 2, such as those described above.

In another aspect, the variant comprises or consists of amino acid substitutions at positions corresponding to positions 127, 341, and 379 of SEQ ID NO: 2, such as those described above.

In another aspect, the variant comprises or consists of amino acid substitutions at positions corresponding to positions 127, 336, and 93 of SEQ ID NO: 2, such as those described above.

In another aspect, the variant comprises or consists of amino acid substitutions at positions corresponding to positions 127, 336, and 97 of SEQ ID NO: 2, such as those described above.

In another aspect, the variant comprises or consists of amino acid substitutions at positions corresponding to positions 127, 336, and 246 of SEQ ID NO: 2, such as those described above.

In another aspect, the variant comprises or consists of amino acid substitutions at positions corresponding to positions 127, 336, and 379 of SEQ ID NO: 2, such as those described above.

In another aspect, the variant comprises or consists of amino acid substitutions at positions corresponding to positions 127, 93, and 97 of SEQ ID NO: 2, such as those described above.

In another aspect, the variant comprises or consists of amino acid substitutions at positions corresponding to positions 127, 93, and 246 of SEQ ID NO: 2, such as those described above.

In another aspect, the variant comprises or consists of amino acid substitutions at positions corresponding to positions 127, 93, and 379 of SEQ ID NO: 2, such as those described above.

In another aspect, the variant comprises or consists of amino acid substitutions at positions corresponding to positions 127, 97, and 246 of SEQ ID NO: 2, such as those described above.

In another aspect, the variant comprises or consists of amino acid substitutions at positions corresponding to positions 127, 97, and 379 of SEQ ID NO: 2, such as those described above.

In another aspect, the variant comprises or consists of amino acid substitutions at positions corresponding to positions 171, 341, and 336 of SEQ ID NO: 2, such as those described above.

In another aspect, the variant comprises or consists of amino acid substitutions at positions corresponding to positions 171, 341, and 93 of SEQ ID NO: 2, such as those described above.

In another aspect, the variant comprises or consists of amino acid substitutions at positions corresponding to positions 171, 341, and 97 of SEQ ID NO: 2, such as those described above.

In another aspect, the variant comprises or consists of amino acid substitutions at positions corresponding to positions 171, 341, and 246 of SEQ ID NO: 2, such as those described above.

In another aspect, the variant comprises or consists of amino acid substitutions at positions corresponding to positions 171, 341, and 379 of SEQ ID NO: 2, such as those described above.

In another aspect, the variant comprises or consists of amino acid substitutions at positions corresponding to positions 171, 336, and 93 of SEQ ID NO: 2, such as those described above.

In another aspect, the variant comprises or consists of amino acid substitutions at positions corresponding to positions 171, 336, and 97 of SEQ ID NO: 2, such as those described above.

In another aspect, the variant comprises or consists of amino acid substitutions at positions corresponding to positions 171, 336, and 246 of SEQ ID NO: 2, such as those described above.

In another aspect, the variant comprises or consists of amino acid substitutions at positions corresponding to positions 171, 336, and 379 of SEQ ID NO: 2, such as those described above.

In another aspect, the variant comprises or consists of amino acid substitutions at positions corresponding to positions 171, 93, and 97 of SEQ ID NO: 2, such as those described above.

In another aspect, the variant comprises or consists of amino acid substitutions at positions corresponding to positions 171, 93, and 246 of SEQ ID NO: 2, such as those described above.

In another aspect, the variant comprises or consists of amino acid substitutions at positions corresponding to positions 171, 93, and 379 of SEQ ID NO: 2, such as those described above.

In another aspect, the variant comprises or consists of amino acid substitutions at positions corresponding to positions 171, 97, and 246 of SEQ ID NO: 2, such as those described above.

In another aspect, the variant comprises or consists of amino acid substitutions at positions corresponding to positions 171, 97, and 379 of SEQ ID NO: 2, such as those described above.

In another aspect, the variant comprises or consists of amino acid substitutions at positions corresponding to positions 171, 246, and 379 of SEQ ID NO: 2, such as those described above.

In another aspect, the variant comprises or consists of amino acid substitutions at positions corresponding to positions 341, 336, and 93 of SEQ ID NO: 2, such as those described above.

In another aspect, the variant comprises or consists of amino acid substitutions at positions corresponding to positions 341, 336, and 97 of SEQ ID NO: 2, such as those described above.

In another aspect, the variant comprises or consists of amino acid substitutions at positions corresponding to positions 341, 336, and 246 of SEQ ID NO: 2, such as those described above.

In another aspect, the variant comprises or consists of amino acid substitutions at positions corresponding to positions 341, 336, and 379 of SEQ ID NO: 2, such as those described above.

In another aspect, the variant comprises or consists of amino acid substitutions at positions corresponding to positions 341, 93, and 97 of SEQ ID NO: 2, such as those described above.

In another aspect, the variant comprises or consists of amino acid substitutions at positions corresponding to positions 341, 93, and 246 of SEQ ID NO: 2, such as those described above.

In another aspect, the variant comprises or consists of amino acid substitutions at positions corresponding to positions 341, 93, and 379 of SEQ ID NO: 2, such as those described above.

In another aspect, the variant comprises or consists of amino acid substitutions at positions corresponding to positions 341, 97, and 246 of SEQ ID NO: 2, such as those described above.

In another aspect, the variant comprises or consists of amino acid substitutions at positions corresponding to positions 341, 97, and 379 of SEQ ID NO: 2, such as those described above.

In another aspect, the variant comprises or consists of amino acid substitutions at positions corresponding to positions 341, 246, and 379 of SEQ ID NO: 2, such as those described above.

In another aspect, the variant comprises or consists of amino acid substitutions at positions corresponding to positions 336, 93, and 97 of SEQ ID NO: 2, such as those described above.

In another aspect, the variant comprises or consists of amino acid substitutions at positions corresponding to positions 336, 93, and 246 of SEQ ID NO: 2, such as those described above.

In another aspect, the variant comprises or consists of amino acid substitutions at positions corresponding to positions 336, 93, and 379 of SEQ ID NO: 2, such as those described above.

In another aspect, the variant comprises or consists of amino acid substitutions at positions corresponding to positions 336, 97, and 246 of SEQ ID NO: 2, such as those described above.

In another aspect, the variant comprises or consists of amino acid substitutions at positions corresponding to positions 336, 97, and 379 of SEQ ID NO: 2, such as those described above.

In another aspect, the variant comprises or consists of amino acid substitutions at positions corresponding to positions 336, 246, and 379 of SEQ ID NO: 2, such as those described above.

In another aspect, the variant comprises or consists of amino acid substitutions at positions corresponding to positions 93, 97, and 246 of SEQ ID NO: 2, such as those described above.

In another aspect, the variant comprises or consists of amino acid substitutions at positions corresponding to positions 93, 97, and 349 of SEQ ID NO: 2, such as those described above.

In another aspect, the variant comprises or consists of amino acid substitutions at positions corresponding to positions 97, 246, and 379 of SEQ ID NO: 2, such as those described above.

In another aspect, the variant comprises or consists of amino acid substitutions at positions corresponding to positions 127, 171, 341, and 336 of SEQ ID NO: 2, such as those described above.

In another aspect, the variant comprises or consists of amino acid substitutions at positions corresponding to positions 127, 171, 341, and 93 of SEQ ID NO: 2, such as those described above.

In another aspect, the variant comprises or consists of amino acid substitutions at positions corresponding to positions 127, 171, 341, and 97 of SEQ ID NO: 2, such as those described above.

In another aspect, the variant comprises or consists of amino acid substitutions at positions corresponding to positions 127, 171, 341, and 246 of SEQ ID NO: 2, such as those described above.

In another aspect, the variant comprises or consists of amino acid substitutions at positions corresponding to positions 127, 171, 341, and 379 of SEQ ID NO: 2, such as those described above.

In another aspect, the variant comprises or consists of amino acid substitutions at positions corresponding to positions 127, 341, 336, and 93 of SEQ ID NO: 2, such as those described above.

In another aspect, the variant comprises or consists of amino acid substitutions at positions corresponding to positions 127, 341, 336, and 97 of SEQ ID NO: 2, such as those described above.

In another aspect, the variant comprises or consists of amino acid substitutions at positions corresponding to positions 127, 341, 336, and 246 of SEQ ID NO: 2, such as those described above.

In another aspect, the variant comprises or consists of amino acid substitutions at positions corresponding to positions 127, 341, 336, and 379 of SEQ ID NO: 2, such as those described above.

In another aspect, the variant comprises or consists of amino acid substitutions at positions corresponding to positions 127, 336, 93, and 97 of SEQ ID NO: 2, such as those described above.

In another aspect, the variant comprises or consists of amino acid substitutions at positions corresponding to positions 127, 336, 93, and 246 of SEQ ID NO: 2, such as those described above.

In another aspect, the variant comprises or consists of amino acid substitutions at positions corresponding to positions 127, 336, 93, and 379 of SEQ ID NO: 2, such as those described above.

In another aspect, the variant comprises or consists of amino acid substitutions at positions corresponding to positions 127, 93, 97, and 246 of SEQ ID NO: 2, such as those described above.

In another aspect, the variant comprises or consists of amino acid substitutions at positions corresponding to positions 127, 93, 97, and 379 of SEQ ID NO: 2, such as those described above.

In another aspect, the variant comprises or consists of amino acid substitutions at positions corresponding to positions 171, 341, 336, and 93 of SEQ ID NO: 2, such as those described above.

In another aspect, the variant comprises or consists of amino acid substitutions at positions corresponding to positions 171, 341, 336, and 97 of SEQ ID NO: 2, such as those described above.

In another aspect, the variant comprises or consists of amino acid substitutions at positions corresponding to positions 171, 341, 336, and 246 of SEQ ID NO: 2, such as those described above.

In another aspect, the variant comprises or consists of amino acid substitutions at positions corresponding to positions 171, 341, 336, and 379 of SEQ ID NO: 2, such as those described above.

In another aspect, the variant comprises or consists of amino acid substitutions at positions corresponding to positions 171, 336, 93, and 97 of SEQ ID NO: 2, such as those described above.

In another aspect, the variant comprises or consists of amino acid substitutions at positions corresponding to positions 171, 336, 93, and 246 of SEQ ID NO: 2, such as those described above.

In another aspect, the variant comprises or consists of amino acid substitutions at positions corresponding to positions 171, 336, 93, and 379 of SEQ ID NO: 2, such as those described above.

In another aspect, the variant comprises or consists of amino acid substitutions at positions corresponding to positions 171, 93, 97, and 246 of SEQ ID NO: 2, such as those described above.

In another aspect, the variant comprises or consists of amino acid substitutions at positions corresponding to positions 171, 93, 97, and 379 of SEQ ID NO: 2, such as those described above.

In another aspect, the variant comprises or consists of amino acid substitutions at positions corresponding to positions 171, 97, 246, and 379 of SEQ ID NO: 2, such as those described above.

In another aspect, the variant comprises or consists of amino acid substitutions at positions corresponding to positions 341, 336, 93, and 97 of SEQ ID NO: 2, such as those described above.

In another aspect, the variant comprises or consists of amino acid substitutions at positions corresponding to positions 341, 336, 93, and 246 of SEQ ID NO: 2, such as those described above.

In another aspect, the variant comprises or consists of amino acid substitutions at positions corresponding to positions 341, 336, 93, and 379 of SEQ ID NO: 2, such as those described above.

In another aspect, the variant comprises or consists of amino acid substitutions at positions corresponding to positions 341, 93, 97, and 246 of SEQ ID NO: 2, such as those described above.

In another aspect, the variant comprises or consists of amino acid substitutions at positions corresponding to positions 341, 93, 97, and 379 of SEQ ID NO: 2, such as those described above.

In another aspect, the variant comprises or consists of amino acid substitutions at positions corresponding to positions 341, 97, 246, and 379 of SEQ ID NO: 2 of SEQ ID NO: 2, such as those described above.

In another aspect, the variant comprises or consists of amino acid substitutions at positions corresponding to positions 93, 97, 246, and 379 of SEQ ID NO: 2, such as those described above.

In another aspect, the variant further comprises or consists of an amino acid substitution at position corresponding to position 363 of SEQ ID NO: 2. In another aspect, the amino acid at a position corresponding to position 363 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala. In another aspect, the variant further comprises or consists of the substitution G363A of the mature polypeptide of SEQ ID NO: 2.

In another aspect, the variant further comprises or consists of an amino acid substitution at position corresponding to position 344 of SEQ ID NO: 2. In another aspect, the amino acid at a position corresponding to position 344 is substituted with Ala, Arg, Asn, Asp, Cys, Gin, Glu, Gly, His, Ile, Leu, Lys, Met, Phe, Pro, Ser, Thr, Trp, Tyr, or Val, preferably with Arg. In another aspect, the variant further comprises or consists of the substitution Q344R of the mature polypeptide of SEQ ID NO: 2.

In another aspect, the variant further comprises or consists of an amino acid substitution at a position corresponding to position 294 of SEQ ID NO: 2. In another aspect, the amino acid at a position corresponding to position 294 is substituted with Ala, Arg, Asn, Asp, Cys, Gin, Glu, Gly, His, Ile, Leu, Lys, Met, Phe, Pro, Ser, Thr, Trp, Tyr, or Val, preferably with Asn. In another aspect, the variant further comprises or consists of the substitution D294N of the mature polypeptide of SEQ ID NO: 2.

In another aspect, the variant comprises or consists of one or more (e.g., several) substitutions selected from the group consisting of T127N, V171I, D341N, Q336E, V93D, V97I, Q246H, and G379C.

In another aspect, the variant comprises or consists of the substitutions T127N+V171I of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions T127N+D341N of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions T127N+D336E of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions V171I+D341N of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions V171I+Q336E of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions D341N+Q336E of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions T127N+V171I+D341N of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions T127N+V171I+Q336E of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions T127N+D341N+Q336E of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions V171I+D341N+Q336E of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions T127N+V171I+D341N+Q336E of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions T127N+V171I+G363A+G379C of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions T127N+V171I Q336E+D341N+G379C of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions T127N+V171I+Q336E+D341N+G363A+G379C of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions V97I+T127N+V171I+Q246H+Q336E+D341N+G379C of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions V97I+T127N+V171I+Q246H+Q336E+D341N+G363A+G379C of the mature polypeptide of SEQ ID NO: 2, and exhibits least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions V93D+V97I+T127N+V171I+Q246H+Q336E+D341N+G379C of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions V93D+V97I+T127N+V171I+Q246H+Q336E+D341N+G363A+G379C of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions V97I+T127N+V171I+Q246H+D294N+Q336E+D341N+Q344R+G363A+G379C of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions V93D+V97I+T127N+V171I+Q246H+D294N+Q336E+D341N+Q344R+G363A+G379C of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

In another aspect, the variant comprises or consists of the substitutions V93D+V97I+T127N+V171I+Q246H+D294N+Q336E+D341N+Q344R+G363A+G379C of the mature polypeptide of SEQ ID NO: 2, and exhibits at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the mature polypeptide of SEQ ID NO: 2 and endoglucanase activity, and further the variant has improved thermostability and/or thermal activity compared to the mature TrEG2 endoglucanase of SEQ ID NO: 2.

Additional mutations, other than those described above, may be introduced into the GH Family 5 endoglucanase, provided that such mutations do not significantly compromise the structure and function of the enzyme. As would be appreciated by those of ordinary skill in the art, but without being limiting in any manner, additional mutations may be introduced in regions of low sequence conservation among GH Family 5 endoglucanases.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for enzyme activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide. Essential amino acids in T. reesei endoglucanase II (EG2 or Cel5A) having the amino acid sequence of SEQ ID NO: 2 include E329 and E218, which serve as the nucleophile and the acid/base respectively and. Other amino acids in T. reesei endoglucanase II (EG2 or Cel5A) having the amino acid sequence of SEQ ID NO: 2 that are highly conserved among Family 5 endoglucanases are R130, H174, N217, H288, and Y290. The variants may consist of 327 to 397 amino acids, e.g., 361 to 397, 327 to 390, and 361 to 390 amino acids, or any number of amino acids therebetween.

In an embodiment, the variant has improved thermal activity compared to the parent endoglucanase. Improved thermal activity of the variant compared to the parent endoglucanase may be determined using the method described in Examples 7 and 8.

In an embodiment, the variant has improved thermostability compared to the parent endoglucanase. Improved thermostability of the variant compared to the parent endoglucanase may be determined using the method described in Examples 7 and 8.

In other embodiments, the variant may be further improved compared to the parent endoglucanase in one or more of the following properties: improved stability under storage conditions, specific activity substrate binding, substrate cleavage, substrate specificity, catalytic efficiency, catalytic rate, pH activity, pH stability, substrate stability, surface properties, chemical stability, or oxidation stability.

### Parent GH Family 5 Endoglucanases

The parent endoglucanase may be (a) a polypeptide having at least 60% sequence identity to the polypeptide of SEQ ID NO: 2; (b) a polypeptide encoded by a polynucleotide that hybridizes under low stringency conditions with (i) the mature polypeptide coding sequence of SEQ ID NO: 1, (ii) the cDNA sequence thereof, or (iii) the full-length complement of (i) or (ii); or (c) a polypeptide encoded by a polynucleotide having at least 60% sequence identity to the mature polypeptide coding sequence of SEQ ID NO: 1.

In an aspect, the parent has a sequence identity to the mature polypeptide of SEQ ID NO: 2 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and which has endoglucanase activity. In one aspect, the amino acid sequence of the parent differs by up to 20 amino acids, e.g., 1, 2, 3, 4, 5, 6, 7, 8, 9, 10, 11, 12, 13, 14, 15, 16, 17, 18, 19 or 20 amino acids from the mature polypeptide of SEQ ID NO: 2.

In another aspect, the parent comprises or consists of the amino acid sequence of SEQ ID NO: 2. In another aspect, the parent comprises or consists of the mature polypeptide of SEQ ID NO: 3. In another aspect, the parent comprises or consists of amino acids 23 to 418 of SEQ ID NO: 3.

In another aspect, the parent is a fragment of the mature polypeptide of SEQ ID NO: 2 containing at least 327 amino acid residues, e.g., at least 361 or at least 390 amino acid residues.

In another embodiment, the parent is an allelic variant of the mature polypeptide of SEQ ID NO: 2.

In another aspect, the parent is encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the cDNA sequence thereof, or (iii) the full-length complement of (i) or (ii) (Sambrook et al., 1989, Molecular Cloning, A Laboratory Manual, 2d edition, Cold Spring Harbor, New York).

The polynucleotide of SEQ ID NO: 1 or a subsequence thereof, as well as the polypeptide of SEQ ID NO: 2 or a fragment thereof, may be used to design nucleic acid probes to identify and clone DNA encoding a parent from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a parent. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1 or a subsequence thereof, the carrier material is used in a Southern blot.

For purposes of the present invention, hybridization indicates that the polynucleotide hybridizes to a labeled nucleic acid probe corresponding to (i) SEQ ID NO: 1; (ii) the mature polypeptide coding sequence of SEQ ID NO: 1; (iii) the cDNA sequence thereof]; (iv) the full-length complement thereof; or (v) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is the mature polypeptide coding sequence of SEQ ID NO: 1. In another aspect, the nucleic acid probe is 325-590 and 765-1692 of SEQ ID NO: 1. In another aspect, the nucleic acid probe is a polynucleotide that encodes the polypeptide of SEQ ID NO: 2; the mature polypeptide thereof; or a fragment thereof. In another aspect, the nucleic acid probe is SEQ ID NO: 1 or the cDNA sequence thereof.

In another embodiment, the parent is encoded by a polynucleotide having a sequence identity to the mature polypeptide coding sequence of SEQ ID NO: 1 or the cDNA sequence thereof of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

The polypeptide may be a hybrid polypeptide in which a region of one polypeptide is fused at the N-terminus or the C-terminus of a region of another polypeptide.

The parent may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent endoglucanase may be obtained from microorganisms of any genus. For purposes of the present invention, the term “obtained from” as used herein in connection with a given source shall mean that the parent endoglucanase encoded by a polynucleotide is produced by the source or by a strain in which the polynucleotide from the source has been inserted. In one aspect, the parent endoglucanase is secreted extracellularly.

The parent may be a bacterial GH Family 5 endoglucanase. For example, the parent may be a Gram-positive bacterial polypeptide such as a Acidothermus, Bacillus, Clostridium, Enterococcus, Geobacillus, Lactobacillus, Lactococcus, Oceanobacillus, Staphylococcus, Streptococcus, or Streptomyces GH Family 5 endoglucanase, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma GH Family 5 endoglucanase

The parent may be a fungal GH Family 5 endoglucanase. For example, the parent may be a yeast GH Family 5 endoglucanase such as a Candida, Kluyveromyces, Pichia, Saccharomyces, Schizosaccharomyces, or Yarrowia GH Family 5 endoglucanase; or a filamentous fungal GH Family 5 endoglucanase such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Hypocrea, Irpex, Lentinula, Leptospaeria, Macrophomina, Magnaporthe, Melanocarpus, Meripilus, Mucor, Myceliophthora, Neocallimastix, Neurospora, Orpinomyces, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria GH Family 5 endoglucanase.

In another aspect, the parent is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Hypocrea jecorina, Humicola insolens, Humicola lanuginosa, Irpex lacteus, Macrophomina phaseolina, Mucor miehei, Myceliophthora thermophila, Neurospora crassa, Penicillium funiculosum, Penicillium janthinefium, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride GH Family 5 endoglucanase.

In another aspect, the parent endoglucanase is a Trichoderma reesei GH Family 5 endoglucanase e.g., the GH Family 5 endoglucanase of SEQ ID NO: 2 or the mature polypeptide thereof.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding a parent may then be obtained by similarly screening a genomic DNA or cDNA library of another microorganism or mixed DNA sample. Once a polynucleotide encoding a parent has been detected with the probe(s), the polynucleotide can be isolated or cloned by utilizing techniques that are known to those of ordinary skill in the art (see, e.g., Sambrook et al., 1989, supra).

### Preparation of Variants

The present invention also relates to methods for obtaining a variant having endoglucanase activity, comprising: (a) introducing into a parent endoglucanase an amino acid substitution at one or more (e.g., several) positions corresponding to positions 127, 171, 341, 336, 93, 97, 246, and 379 of the mature polypeptide of SEQ ID NO: 2, wherein the variant has endoglucanase activity; and (b) recovering the variant.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to polynucleotides encoding a GH Family 5 endoglucanase variant of the present invention.

### Nucleic Acid Constructs

The present invention also relates to nucleic acid constructs comprising a polynucleotide encoding a GH Family 5 endoglucanase variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide which is recognized by a host cell for expression of the polynucleotide. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator sequence is operably linked to the 3′-terminus of the polynucleotide encoding the variant. Any terminator that is functional in the host cell may be used.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader sequence is operably linked to the 5′-terminus of the polynucleotide encoding the variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the variant-encoding sequence and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, and Trichoderma reesei beta-xylosidase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of the variant and the signal peptide sequence is positioned next to the N-terminus of the propeptide sequence.

It may also be desirable to add regulatory sequences that regulate expression of the variant relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, Aspergillus oryzae glucoamylase promoter, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked with the regulatory sequence.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide encoding a variant of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804) or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Hypocrea, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Hypocrea jecorina, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Fungal cells may also be transformed by a biolistic process involving introduction of gold or tungsten pellets coated with one or more polynucleotides into fungal spores using a particle gun. Suitable methods for biolistic transformation of Trichoderma host cells are described in U.S. Publication No. 2013/0052694.

Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a variant, comprising: (a) cultivating a host cell of the present invention under conditions suitable for expression of the variant; and (b) recovering the variant.

The host cells are cultivated in a nutrient medium suitable for production of the variant using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the variants. These detection methods include, but are not limited to, use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, collection, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

### Plants

The present invention also relates to plants, e.g., a transgenic plant, plant part, or plant cell, comprising a polynucleotide of the present invention so as to express and produce the variant in recoverable quantities. The variant may be recovered from the plant or plant part. Alternatively, the plant or plant part containing the variant may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats.

Also included within the scope of the present invention are the progeny of such plants, plant parts, and plant cells.

The transgenic plant or plant cell expressing a variant may be constructed in accordance with methods known in the art. In short, the plant or plant cell is constructed by incorporating one or more expression constructs encoding a variant into the plant host genome or chloroplast genome and propagating the resulting modified plant or plant cell into a transgenic plant or plant cell.

The expression construct is conveniently a nucleic acid construct that comprises a polynucleotide encoding a variant operably linked with appropriate regulatory sequences required for expression of the polynucleotide in the plant or plant part of choice. Furthermore, the expression construct may comprise a selectable marker useful for identifying plant cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences, is determined, for example, on the basis of when, where, and how the variant is desired to be expressed. For instance, the expression of the gene encoding a variant may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, or the rice actin 1 promoter may be used (Franck et al., 1980, Cell 21: 285-294; Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant Cell Physiol. 39: 885-889), a Vicia faba promoter from the legumin B4 and the unknown seed protein gene from Vicia faba (Conrad et al., 1998, J. Plant Physiol. 152: 708-711), a promoter from a seed oil body protein (Chen et al., 1998, Plant Cell Physiol. 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiol. 102: 991-1000), the chlorella virus adenine methyltransferase gene promoter (Mitra and Higgins, 1994, Plant Mol. Biol. 26: 85-93), the aldP gene promoter from rice (Kagaya et al., 1995, Mol. Gen. Genet. 248: 668-674), or a wound inducible promoter such as the potato pin2 promoter (Xu et al., 1993, Plant Mol. Biol. 22: 573-588). Likewise, the promoter may be induced by abiotic treatments such as temperature, drought, or amino acid substitutions in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a variant in the plant. For instance, the promoter enhancer element may be an intron that is placed between the promoter and the polynucleotide encoding a variant. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Agrobacterium tumefaciens-mediated gene transfer is a method for generating transgenic dicots (for a review, see Hooykas and Schilperoort, 1992, Plant Mol. Biol. 19: 15-38) and for transforming monocots, although other transformation methods may be used for these plants. A method for generating transgenic monocots is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al., 1992, Bio/Technology 10: 667-674). An alternative method for transformation of monocots is based on protoplast transformation as described by Omirulleh et al., 1993, Plant Mol. Biol. 21: 415-428. Additional transformation methods include those described in U.S. Pat. Nos. 6,395,966 and 7,151,204 (both of which are herein incorporated by reference in their entirety).

Following transformation, the transformants having incorporated the expression construct are selected and regenerated into whole plants according to methods well known in the art. Often the transformation procedure is designed for the selective elimination of selection genes either during regeneration or in the following generations by using, for example, co-transformation with two separate T-DNA constructs or site specific excision of the selection gene by a specific recombinase.

In addition to direct transformation of a particular plant genotype with a construct of the present invention, transgenic plants may be made by crossing a plant having the construct to a second plant lacking the construct. For example, a construct encoding a variant can be introduced into a particular plant variety by crossing, without the need for ever directly transforming a plant of that given variety. Therefore, the present invention encompasses not only a plant directly regenerated from cells which have been transformed in accordance with the present invention, but also the progeny of such plants. As used herein, progeny may refer to the offspring of any generation of a parent plant prepared in accordance with the present invention. Such progeny may include a DNA construct prepared in accordance with the present invention. Crossing results in the introduction of a transgene into a plant line by cross pollinating a starting line with a donor plant line. Non-limiting examples of such steps are described in U.S. Pat. No. 7,151,204.

Plants may be generated through a process of backcross conversion. For example, plants include plants referred to as a backcross converted genotype, line, inbred, or hybrid.

Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

The present invention also relates to methods of producing a variant of the present invention comprising: (a) cultivating a transgenic plant or a plant cell comprising a polynucleotide encoding the variant under conditions conducive for production of the variant; and (b) recovering the variant.

### Compositions

The present invention also relates to compositions comprising a polypeptide of the present invention.

The compositions may comprise a polypeptide of the present invention as the major enzymatic component, e.g., a mono-component composition.

Alternatively, the compositions may comprise multiple enzymatic activities, such as one or more (e.g., several) enzymes selected from the group consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, and xylanase; in particular a composition comprising a xylanase and an endoglucanase variant according to the invention.

The compositions may be prepared in accordance with methods known in the art and may be in the form of a liquid or a dry composition. The compositions may be stabilized in accordance with methods known in the art.

Examples are given below of preferred uses of the compositions of the present invention. The dosage of the composition and other conditions under which the composition is used may be determined on the basis of methods known in the art.

### Uses

Brewing:

The use of enzymes in brewing is common. Application of enzymes to the mashing step to improve mash filterability and increase extract yield is known in the art. However, there is a need for improvement of the filtration step; in particular by reducing the viscosity of the mash.

It may be advantageous to add a GH Family 5 endoglucanase variant according to the invention to a mash in order to reduce the viscosity of the mash.

Biomass:

The present invention also relates to processes for degrading a cellulosic or hemicellulosic material, comprising: treating the cellulosic or hemicellulosic material with an enzyme composition comprising a GH Family 5 endoglucanase variant according to the invention.

In one aspect, the processes further comprise recovering the degraded cellulosic or hemicellulosic material. Soluble products from the degradation of the cellulosic or hemicellulosic material can be separated from insoluble cellulosic or hemicellulosic material using methods known in the art such as, for example, centrifugation, filtration, or gravity settling.

The present invention also relates to processes of producing a fermentation product, comprising:

(a) saccharifying a cellulosic or hemicellulosic material with an enzyme composition comprising a GH Family 5 endoglucanase variant according to the invention;

(b) fermenting the saccharified cellulosic or hemicellulosic material with one or more (e.g., several) fermenting microorganisms to produce the fermentation product; and

(c) recovering the fermentation product from the fermentation.

The present invention also relates to processes of fermenting a cellulosic or hemicellulosic material, comprising: fermenting the cellulosic or hemicellulosic material with one or more (e.g., several) fermenting microorganisms, wherein the cellulosic or hemicellulosic material is saccharified with an enzyme composition comprising a GH Family 5 endoglucanase variant according to the invention. In one aspect, the fermenting of the cellulosic or hemicellulosic material produces a fermentation product. In another aspect, the processes further comprise recovering the fermentation product from the fermentation.

The processes of the present invention can be used to saccharify the cellulosic or hemicellulosic material to fermentable sugars and to convert the fermentable sugars to many useful fermentation products, e.g., fuel (ethanol, n-butanol, isobutanol, biodiesel, jet fuel) and/or platform chemicals (e.g., acids, alcohols, ketones, gases, oils, and the like). The production of a desired fermentation product from the cellulosic or hemicellulosic material typically involves pretreatment, enzymatic hydrolysis (saccharification), and fermentation.

The processing of the cellulosic or hemicellulosic material according to the present invention can be accomplished using methods conventional in the art. Moreover, the processes of the present invention can be implemented using any conventional biomass processing apparatus configured to operate in accordance with the invention.

Hydrolysis (saccharification) and fermentation, separate or simultaneous, include, but are not limited to, separate hydrolysis and fermentation (SHF); simultaneous saccharification and fermentation (SSF); simultaneous saccharification and co-fermentation (SSCF); hybrid hydrolysis and fermentation (HHF); separate hydrolysis and co-fermentation (SHCF); hybrid hydrolysis and co-fermentation (HHCF); and direct microbial conversion (DMC), also sometimes called consolidated bioprocessing (CBP). SHF uses separate process steps to first enzymatically hydrolyze the cellulosic or hemicellulosic material to fermentable sugars, e.g., glucose, cellobiose, and pentose monomers, and then ferment the fermentable sugars to ethanol. In SSF, the enzymatic hydrolysis of the cellulosic or hemicellulosic material and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF involves the co-fermentation of multiple sugars (Sheehan and Himmel, 1999, Biotechnol. Prog. 15: 817-827). HHF involves a separate hydrolysis step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation strain can tolerate. DMC combines all three processes (enzyme production, hydrolysis, and fermentation) in one or more (e.g., several) steps where the same organism is used to produce the enzymes for conversion of the cellulosic or hemicellulosic material to fermentable sugars and to convert the fermentable sugars into a final product (Lynd et al., 2002, Microbiol. Mol. Biol. Reviews 66: 506-577). It is understood herein that any method known in the art comprising pretreatment, enzymatic hydrolysis (saccharification), fermentation, or a combination thereof, can be used in the practicing the processes of the present invention.

A conventional apparatus can include a fed-batch stirred reactor, a batch stirred reactor, a continuous flow stirred reactor with ultrafiltration, and/or a continuous plug-flow column reactor (de Castilhos Corazza et al., 2003, Acta Scientiarum. Technology 25: 33-38; Gusakov and Sinitsyn, 1985, Enz. Microb. Technol. 7: 346-352), an attrition reactor (Ryu and Lee, 1983, Biotechnol. Bioeng. 25: 53-65). Additional reactor types include fluidized bed, upflow blanket, immobilized, and extruder type reactors for hydrolysis and/or fermentation.

The invention is further defined by the following paragraphs:

Paragraph 1. A variant of GH Family 5 endoglucanases, comprising a substitution at one or more (e.g., several) positions corresponding to positions 127, 171, 341, 336, 93, 97, 246, and 379 of SEQ ID NO: 2, wherein the variant has endoglucanase activity, and wherein the variant has at least 80%, e.g., at least 85%, at least 90%, at least 91%, at least 92%, at least 93%, at least 94%, at least 95%, at least 96%, at least 97%, at least 98%, or at least 99%, but less than 100%, to the amino acid 71-397 of SEQ ID NO:2.

Paragraph 2. A variant of GH Family 5 endoglucanases, comprising a substitution at one or more (e.g., several) positions corresponding to positions 127, 171, 341, 336, 93, 97, 246, and 379 of the mature polypeptide of SEQ ID NO: 2, wherein the variant has endoglucanase activity and wherein the variant has sequence identity of at least 80%, e.g., at least 85%, at least 90%, at least 91%, at least 92%, at least 93%, at least 94%, at least 95%, at least 96%, at least 97%, at least 98%, or at least 99%, but less than 100%, to the amino acid sequence 71-397 of SEQ ID NO:2.

Paragraph 3. A variant of GH Family 5 endoglucanases, comprising a substitution at one or more (e.g., several) positions corresponding to positions 127, 171, 341, 336, 93, 97, 246, and 379 of the mature polypeptide of SEQ ID NO: 2, wherein the variant has endoglucanase activity and wherein the variant has sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent endoglucanase of SEQ ID NO:2 or to amino acids 23 to 418 of SEQ ID NO: 3.

Paragraph 4. The variant of any of paragraphs 1 to 3, which is a variant of a parent GH Family 5 endoglucanase selected from the group consisting of:
a. a polypeptide having at least 60% sequence identity to amino acids 71-397 of SEQ ID NO: 2;
b. a polypeptide encoded by a polynucleotide that hybridizes under at least low stringency conditions with (i) the mature polypeptide coding sequence of SEQ ID NO: 1, (ii) the cDNA sequence thereof, or (iii) the full-length complement of (i) or (ii);
c. a polypeptide encoded by a polynucleotide having at least 60%, identity to the mature polypeptide coding sequence of SEQ ID NO: 1 or the cDNA sequence thereof; and
d. a fragment of SEQ ID NO: 2, which has endoglucanase activity.

Paragraph 5. A variant of GH Family 5 endoglucanases, comprising a substitution at one or more (e.g., several) positions corresponding to positions 127, 171, 341, 336, 93, 97, 246, and 379 of the mature polypeptide of SEQ ID NO: 2, wherein the variant has endoglucanase activity.

Paragraph 6. A variant of GH Family 5 endoglucanases, comprising a substitution at one or more (e.g., several) positions corresponding to positions 127, 171, 341, 336, 93, 97, 246, and 379 of the mature polypeptide of SEQ ID NO: 2, wherein the variant has endoglucanase activity and wherein the variant has sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent endoglucanase.

Paragraph 7. A variant of GH Family 5 endoglucanases, comprising a substitution at one or more (e.g., several) positions corresponding to positions 127, 171, 341, 336, 93, 97, 246, and 379 of the mature polypeptide of SEQ ID NO: 2, wherein the variant has endoglucanase activity and wherein the variant has sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent endoglucanase of SEQ ID NO:2 or to amino acids 23 to 418 of SEQ ID NO: 3.

Paragraph 8. The variant of any of paragraphs 1 to 3, which is a variant of a parent GH Family 5 endoglucanase selected from the group consisting of:

a. a polypeptide having at least 60% sequence identity to amino acids 71-397 of SEQ ID NO: 2;

b. a polypeptide encoded by a polynucleotide that hybridizes under at least low stringency conditions with (i) the mature polypeptide coding sequence of SEQ ID NO: 1, (ii) the cDNA sequence thereof, or (iii) the full-length complement of (i) or (ii);

c. a polypeptide encoded by a polynucleotide having at least 60%, identity to the mature polypeptide coding sequence of SEQ ID NO: 1 or the cDNA sequence thereof; and

d. a fragment of SEQ ID NO: 2, which has endoglucanase activity.

Paragraph 9. The variant of any of paragraphs 2 to 4 wherein the parent GH Family 5 endoglucanase has at least 60%, e.g., at least 65%, at least 70%, at least 75%, at least 80%, at least 85%, at least 90%, at least 95%, at least 96%, at least 97%, at least 98%, at least 99% or 100% sequence identity to amino acids 71-397 of SEQ ID NO: 2.

Paragraph 10. The variant of any of paragraphs 2 to 5 wherein the parent GH Family 5 endoglucanase is a polypeptide encoded by a polynucleotide that hybridizes under at least low stringency conditions with (i) the mature polypeptide coding sequence of SEQ ID NO: 1, (ii) the cDNA sequence thereof, or (iii) the full-length complement of (i) or (ii).

Paragraph 11. The variant of any of paragraphs 2 to 5 wherein the parent GH Family 5 endoglucanase is a polypeptide encoded by a polynucleotide that hybridizes under at least medium stringency conditions with (i) the mature polypeptide coding sequence of SEQ ID NO: 1, (ii) the cDNA sequence thereof, or (iii) the full-length complement of (i) or (ii).

Paragraph 12. The variant of any of paragraphs 2 to 5 wherein the parent GH Family 5 endoglucanase is a polypeptide encoded by a polynucleotide that hybridizes under at least high stringency conditions with (i) the mature polypeptide coding sequence of SEQ ID NO: 1, (ii) the cDNA sequence thereof, or (iii) the full-length complement of (i) or (ii).

Paragraph 13. The variant of any of paragraphs 2 to 5 wherein the parent GH Family 5 endoglucanase is a polypeptide encoded by a polynucleotide that hybridizes under at least very high stringency conditions with (i) the mature polypeptide coding sequence of SEQ ID NO: 1, (ii) the cDNA sequence thereof, or (iii) the full-length complement of (i) or (ii).

Paragraph 14. The variant of any of paragraphs 2 to 5 wherein the parent GH Family 5 endoglucanase is a polypeptide encoded by a polynucleotide having at least 60%, e.g., at least 65%, at least 70%, at least 75%, at least 80%, at least 85%, at least 90%, at least 95%, at least 96%, at least 97%, at least 98%, at least 99% or 100%, identity to the mature polypeptide coding sequence of SEQ ID NO: 1 or the cDNA sequence thereof.

Paragraph 15. The variant of any of paragraphs 2 to 5 wherein the parent GH Family 5 endoglucanase is a fragment of SEQ ID NO: 2 having endoglucanase activity.

Paragraph 16. The variant of paragraph 15, wherein the fragment of SEQ ID NO: 2 comprises or consists of at least 361 amino acid residues.

Paragraph 17. The variant of paragraph 15, wherein the fragment of SEQ ID NO: 2 comprises or consists of at least 327 amino acid residues.

Paragraph 18. The variant of paragraph 15, wherein the fragment of SEQ ID NO: 2 comprises or consists of at least 310 amino acid residues.

Paragraph 19. The variant of paragraph 15, wherein the fragment of SEQ ID NO: 2 comprises or consists of amino acids 37 to 397 of SEQ ID NO: 2.

Paragraph 20. The variant of paragraph 15, wherein the fragment of SEQ ID NO: 2 comprises or consists of amino acids 71 to 391 of SEQ ID NO: 2.

Paragraph 21. The variant of paragraph 15, wherein the fragment of SEQ ID NO: 2 comprises or consists of, amino acids 81 to 390 of SEQ ID NO: 2.

Paragraph 22. The variant of any of paragraphs 1 to 21, wherein the number of amino acid substitutions is 1-20, e.g., 1-10 or 1-5, such as 1, 2, 3, 4, 5, 6, 7, 8, 9, 10, 11, 12, 13, 14, 15, 16, 17, 18, 19, or 20, amino acid substitutions.

Paragraph 23. The variant of any of paragraphs 1 to 18, comprising an amino acid substitution at two positions corresponding to any of positions 127, 171, 341, 336, 93, 97, 246, and 379 of SEQ ID NO: 2.

Paragraph 24. The variant of any of paragraphs 1 to 18, comprising an amino acid substitution at three positions corresponding to any of positions 127, 171, 341, 336, 93, 97, 246, and 379 of SEQ ID NO: 2.

Paragraph 25. The variant of any of paragraphs 1 to 18, comprising an amino acid substitution at four positions corresponding to any of positions 127, 171, 341, 336, 93, 97, 246, and 379 of SEQ ID NO: 2.

Paragraph 26. The variant of any of paragraphs 1 to 18, comprising an amino acid substitution at five positions corresponding to any of positions 127, 171, 341, 336, 93, 97, 246, and 379 of SEQ ID NO: 2.

Paragraph 27. The variant of any of paragraphs 1 to 18, comprising an amino acid substitution at six positions corresponding to any of positions 127, 171, 341, 336, 93, 97, 246, and 379 of SEQ ID NO: 2.

Paragraph 28. The variant of any of paragraphs 1 to 18, comprising an amino acid substitution at seven positions corresponding to any of positions 127, 171, 341, 336, 93, 97, 246, and 379 of SEQ ID NO: 2.

Paragraph 29. The variant of any of paragraphs 1 to 18, comprising an amino acid substitution at each position corresponding to positions 127, 171, 341, 336, 93, 97, 246, and 379 of SEQ ID NO: 2.

Paragraph 30. The variant of any of paragraphs 1 to 18, wherein the amino acid at a position corresponding to position 127 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile.

Paragraph 31. The variant of any of paragraphs 1 to 18, which comprises or consists of the substitution T127N of the mature polypeptide of SEQ ID NO: 2.

Paragraph 32. The variant of any of paragraphs 1 to 18, wherein the amino acid at a position corresponding to position 171 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile.

Paragraph 33. The variant of any of paragraphs 1 to 18, which comprises or consists of the substitution V171I of the mature polypeptide of SEQ ID NO: 2.

Paragraph 34. The variant of any of paragraphs 1 to 18, wherein the amino acid at a position corresponding to position 341 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile.

Paragraph 35. The variant of any of paragraphs 1 to 18, which comprises or consists of the substitution D341N of the mature polypeptide of SEQ ID NO: 2.

Paragraph 36. The variant of any of paragraphs 1 to 18, wherein the amino acid at a position corresponding to position 336 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile.

Paragraph 37. The variant of any of paragraphs 1 to 18, which comprises or consists of the substitution Q336E of the mature polypeptide of SEQ ID NO: 2.

Paragraph 38. The variant of any of paragraphs 1 to 18, wherein the amino acid at a position corresponding to position 93 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile.

Paragraph 39. The variant of any of paragraphs 1 to 18, which comprises or consists of the substitution V93D of the mature polypeptide of SEQ ID NO: 2.

Paragraph 40. The variant of any of paragraphs 1 to 18, wherein the amino acid at a position corresponding to position 97 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile.

Paragraph 41. The variant of any of paragraphs 1 to 18, which comprises or consists of the substitution V97I of the mature polypeptide of SEQ ID NO: 2.

Paragraph 42. The variant of any of paragraphs 1 to 18, wherein the amino acid at a position corresponding to position 246 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile.

Paragraph 43. The variant of any of paragraphs 1 to 18, which comprises or consists of the substitution Q246H of the mature polypeptide of SEQ ID NO: 2.

Paragraph 44. The variant of any of paragraphs 1 to 18, wherein the amino acid at a position corresponding to position 379 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile.

Paragraph 45. The variant of any of paragraphs 1 to 18, which comprises or consists of the substitution G379C of SEQ ID NO: 2.

Paragraph 46. The variant of any of paragraphs 1 to 18, which comprises one or more substitutions selected from the group consisting of T127N, V171I, D341N, Q336E, V93D, V97I, Q246H, and G379C.

Paragraph 47. The variant of any of paragraphs 1 to 18, which comprises two or more substitutions selected from the group consisting of T127N, V171I, D341N, Q336E, V93D, V97I, Q246H, and G379C.

Paragraph 48. The variant of paragraph 47, wherein the two substitutions are selected from the group consisting of: T127N and V171I; T127N and D341N; T127N and Q336E; T127N and V93D; T127N and V97I; T127N and Q246H; T127N and G379C; V171I and D341N; V171I and Q336E; V171I and V93D; V171I and V97I; V171I and Q246H; V171I and G379C; D341N and Q336E; D341N and V93D; D341N and V97I; D341N and Q246H; D341N and G379C; Q336E and V93D; Q336E and V97I; Q336E and Q246H; Q336E and G379C; V93D and V97I; V93D and Q246H; V93D and G379C; V97I and Q246H; V97I and G379C; Q246H and G379C.

Paragraph 49. The variant of any of paragraphs 1 to 18, which comprises three or more substitutions selected from the group consisting of T127N, V171I, D341N, Q336E, V93D, V97I, Q246H, and G379C.

Paragraph 50. The variant of paragraph 49, wherein the three substitutions are selected from the group consisting of: T127N, V171I, and D341N; T127N, V171I, and Q336E; T127N, V171I, and V93D; T127N, V171I, and V97I; T127N, V171I, and Q246H; T127N, V171I, and G379C; T127N, D341N, and Q336E; T127N, D341N, and V93D; T127N, D341N, and V97I; T127N, D341N, and Q246H; T127N, D341N, and G379C; T127N, Q336E, and V93D; T127N, Q336E, and V97I; T127N, Q336E, and Q246H; T127N, Q336E, and G379C; T127N, V93D, and V97I; T127N, V93D, and Q246H; T127N, V93D, and G379C; T127N, V97I, and Q246H; T127N, V97I, and G379C; T127N, Q246H, and G379C; V171I, D341N, and Q336E; V171I, D341N, and V93D; V171I, D341N, and V97I; V171I, D341N, and Q246H; V171I, D341N, and G379C; V171I, Q336E, and V93D; V171I, Q336E, and V97I; V171I, Q336E, and Q246H; V171I, Q336E, and G379C; V171I, V93D, and V97I; V171I, V93D, and Q246H; V171I, V93D, and G379C; V171I, V97I, and Q246H; V171I, V97I, and G379C; V171I, Q246H, and G379C; D341N, Q336E, and V93D; D341N, Q336E, and V97I; D341N, Q336E, and Q246H; D341N, Q336E, and G379C; D341N, V93D, and V97I; D341N, V93D, and Q246H; D341N, V93D, and G379C; D341N, V97I, and Q246H; D341N, V97I, and G379C; D341N, Q246H, and G379C; Q336E, V93D, and V97I; Q336E, V93D, and Q246H; Q336E, V93D, and G379C; Q336E, V97I, and Q246H; Q336E, V97I, and G379C; Q336E, Q246H, and G379C; V93D, V97I, and Q246H; V93D, V97I, and G379C; V93D, Q246H, and G379C; V97I, Q246H, and G379C.

Paragraph 51. The variant of any of paragraphs 1 to 18, which comprises four or more substitutions selected from the group consisting of T127N, V171I, D341N, Q336E, V93D, V97I, Q246H, and G379C.

Paragraph 52. The variant of paragraph 51, wherein the four substitutions are selected from the group consisting of: T127N, V171I, D341N, and Q336E; T127N, V171I, D341N, and V93D; T127N, V171I, D341N, and V97I; T127N, V171I, D341N, and Q246H; T127N, V171I, D341N, and G379C; T127N, V171I, Q336E, and V93D; T127N, V171I, Q336E, and V97I; T127N, V171I, Q336E, and Q246H; T127N, V171I, Q336E, and G379C; T127N, V171I, V93D, and V97I; T127N, V171I, V93D, and Q246H; T127N, V171I, V93D, and G379C; T127N, V171I, V97I, and Q246H; T127N, V171I, V97I, and G379C; T127N, V171I, Q246H, and G379C; T127N, D341N, Q336E, and V93D; T127N, D341N, Q336E, and V97I; T127N, D341N, Q336E, and Q246H; T127N, D341N, Q336E, and G379C; T127N, D341N, V93D, and V97I; T127N, D341N, V93D, and Q246H; T127N, D341N, V93D, and G379C; T127N, D341N, V97I, and Q246H; T127N, D341N, V97I, and G379C; T127N, D341N, Q246H, and G379C; T127N, Q336E, V93D, and V97I; T127N, Q336E, V93D, and Q246H; T127N, Q336E, V93D, and G379C; T127N, Q336E, V97I, and Q246H; T127N, Q336E, V97I, and G379C; T127N, Q336E, Q246H, and G379C; T127N, V93D, V97I, and Q246H; T127N, V93D, V97I, and G379C; T127N, V93D, Q246H, and G379C; T127N, V97I, Q246H, and G379C; V171I, D341N, Q336E, and V93D; V171I, D341N, Q336E, and V97I; V171I, D341N, Q336E, and Q246H; V171I D341N, Q336E, and G379C; V171I, D341N, V93D, and V97I; V171I, D341N, V93D, and Q246H; V171I, D341N, V93D, and G379C; V171I, D341N, V97I, and Q246H; V171I, D341N, V97I, and G379C; V171I, D341N, Q246H, and G379C; V171I, Q336E, V93D, and V97I; V171I, Q336E, V93D, and Q246H; V171I, Q336E, V93D, and G379C; V171I, Q336E, V97I, and Q246H; V171I, Q336E, V97I, and G379C; V171I, Q336E, Q246H, and G379C; V171I, V93D, V97I, and Q246H; V171I, V93D, V97I, and G379C; V171I, V93D, Q246H, and G379C; V171I, V97I, Q246H, and G379C; D341N, Q336E, V93D, and V97I; D341N, Q336E, V93D, and Q246H; D341N, Q336E, V93D, and G379C; D341N, Q336E, V97I, and Q246H; D341N, Q336E, V97I, and G379C; D341N, Q336E, Q246H, and G379C; D341N, V93D, V97I, and Q246H; D341N, V93D, V97I, and G379C; D341N, V93D, Q246H, and G379C; D341N, V97I, Q246H, and G379C; Q336E, V93D, V97I, and Q246H; Q336E, V93D, V97I, and G379C; Q336E, V93D, Q246H, and G379C; Q336E, V97I, Q246H, and G379C; V93D, V97I, Q246H, and G379C.

Paragraph 53. The variant of any of paragraphs 1 to 18, which comprises five or more substitutions selected from the group consisting of T127N, V171I, D341N, Q336E, V93D, V97I, Q246H, and G379C.

Paragraph 54. The variant of paragraph 53, wherein the five substitutions are selected from the group consisting of: T127N, V171I, D341N, Q336E, and V93D; T127N, V171I, D341N, Q336E, and V97I; T127N, V171I, D341N, Q336E, and Q246H; T127N, V171I, D341N, Q336E, and G379C; T127N, V171I, D341N, V93D, and V97I; T127N, V171I, D341N, V93D, and Q246H; T127N, V171I, D341N, V93D, and G379C; T127N, V171I, D341N, V97I, and Q246H; T127N, V171I, D341N, V97I, and G379C; T127N, V171I, D341N, Q246H, and G379C; T127N, V171I, Q336E, V93D, and V97I; T127N, V171I, Q336E, V93D, and Q246H; T127N, V171I, Q336E, V93D, and G379C; T127N, V171I, Q336E, V97I, and Q246H; T127N, V171I, Q336E, V97I, and G379C; T127N, V171I, Q336E, Q246H, and G379C; T127N, V171I, V93D, V97I, and Q246H; T127N, V171I, V93D, V97I, and G379C; T127N, V171I, V93D, Q246H, and G379C; T127N, V171I, V97I, Q246H, and G379C; T127N, D341N, Q336E, V93D, and V97I; T127N, D341N, Q336E, V93D, and Q246H; T127N, D341N, Q336E, V93D, and G379C; T127N, D341N, Q336E, V97I, and Q246H; T127N, D341N, Q336E, V97I, and G379C; T127N, D341N, Q336E, Q246H, and G379C; T127N, D341N, V93D, V97I, and Q246H; T127N, D341N, V93D, V97I, and G379C; T127N, D341N, V93D, Q246H, and G379C; T127N, D341N, V97I, Q246H, and G379C; T127N, Q336E, V93D, V97I, and Q246H; T127N, Q336E, V93D, V97I, and G379C; T127N, Q336E, V93D, Q246H, and G379C; T127N, Q336E, V97I, Q246H, and G379C; T127N, V93D, V97I, Q246H, and G379C; V171I, D341N, Q336E, V93D, and V97I; V171I, D341N, Q336E, V93D, and Q246H; V171I, D341N, Q336E, V93D, and G379C; V171I, D341N, Q336E, V97I, and Q246H; V171I, D341N, Q336E, V97I, and G379C; V171I, D341N, Q336E, Q246H, and G379C; V171I, D341N, V93D, V97I, and Q246H; V171I, D341N, V93D, V97I, and G379C; V171I, D341N, V93D, Q246H, and G379C; V171I, D341N, V97I, Q246H, and G379C; V171I, Q336E, V93D, V97I, and Q246H; V171I, Q336E, V93D, V97I, and G379C; V171I, Q336E, V93D, Q246H, and G379C; V171I, Q336E, V97I, Q246H, and G379C; V171I, V93D, V97I, Q246H, and G379C; D341N, Q336E, V93D, V97I, and Q246H; D341N, Q336E, V93D, V97I, and G379C; D341N, Q336E, V93D, Q246H, and G379C; D341N, Q336E, V97I, Q246H, and G379C; D341N, V93D, V97I, Q246H, and G379C; Q336E, V93D, V97I, Q246H, and G379C.

Paragraph 55. The variant of any of paragraphs 1 to 18, which comprises six or more substitutions selected from the group consisting of T127N, V171I, D341N, Q336E, V93D, V97I, Q246H, and G379C.

Paragraph 56. The variant of paragraph 55, wherein the six substitutions are selected from the group consisting of: T127N, V171I, D341N, Q336E, V93D, and V97I; T127N, V171I, D341N, Q336E, V93D, and Q246H; T127N, V171I, D341N, Q336E, V93D, and G379C; T127N, V171I, D341N, Q336E, V97I, and Q246H; T127N, V171I, D341N, Q336E, V97I, and G379C; T127N, V171I, D341N, Q336E, Q246H, and G379C; T127N, V171I, D341N, V93D, V97I, and Q246H; T127N, V171I, D341N, V93D, V97I, and G379C; T127N, V171I, D341N, V93D, Q246H, and G379C; T127N, V171I, D341N, V97I, Q246H, and G379C; T127N, V171I, Q336E, V93D, V97I, and Q246H; T127N, V171I, Q336E, V93D, V97I, and G379C; T127N, V171I, Q336E, V93D, Q246H, and G379C; T127N, V171I, Q336E, V97I, Q246H, and G379C; T127N, V171I, V93D, V97I, Q246H, and G379C; T127N, D341N, Q336E, V93D, V97I, and Q246H; T127N, D341N, Q336E, V93D, V97I, and G379C; T127N, D341N, Q336E, V93D, Q246H, and G379C; T127N, D341N, Q336E, V97I, Q246H, and G379C; T127N, D341N, V93D, V97I, Q246H, and G379C; T127N, Q336E, V93D, V97I, Q246H, and G379C; V171I, D341N, Q336E, V93D, V97I, and Q246H; V171I, D341N, Q336E, V93D, V97I, and G379C; V171I, D341N, Q336E, V93D, Q246H, and G379C; V171I, D341N, Q336E, V97I, Q246H, and G379C; V171I D341N, V93D, V97I, Q246H, and G379C; V171I, Q336E, V93D, V97I, Q246H, and G379C; D341N, Q336E, V93D, V97I, Q246H, and G379C.

Paragraph 57. The variant of any of paragraphs 1 to 18, which comprises seven or more substitutions selected from the group consisting of T127N, V171I, D341N, Q336E, V93D, V97I, Q246H, and G379C.

Paragraph 58. The variant of paragraph 57, wherein the seven substitutions are selected from the group consisting of: T127N, V171I, D341N, Q336E, V93D, V97I, and Q246H; T127N, V171I, D341N, Q336E, V93D, V97I, and G379C; T127N, V171I, D341N, Q336E, V93D, Q246H, and G379C; T127N, V171I, D341N, Q336E, V97I, Q246H, and G379C; T127N, V171I, D341N, V93D, V97I, Q246H, and G379C; T127N, V171I, Q336E, V93D, V97I, Q246H, and G379C; T127N, D341N, Q336E, V93D, V97I, Q246H, and G379C; V171I, D341N, Q336E, V93D, V97I, Q246H, and G379C.

Paragraph 59. The variant of any of paragraphs 1 to 18, which comprises amino acid substitutions T127N, V171I, D341N, Q336E, V93D, V97I, Q246H, and G379C.

Paragraph 60. The variant of any of paragraphs 1-59, wherein the amino acid at a position corresponding to position 363 is substituted with Ala, Arg, Asn, Asp, Cys, Gin, Glu, Gly, His, Ile, Leu, Lys, Met, Phe, Pro, Ser, Thr, Trp, Tyr, or Val, preferably with Ala.

Paragraph 61. The variant of any of paragraphs 1-59, wherein variant further comprises or consists of the substitution G363A of the mature polypeptide of SEQ ID NO: 2.

Paragraph 62. The variant of paragraph 61, wherein the variant comprises amino acid substitutions T127N, V171I, G363A, and G379C.

Paragraph 63. The variant of paragraph 61, wherein the variant comprises amino acid substitutions T127N, V171I, Q336E, D341N, G363A, and G379C.

Paragraph 64. The variant of paragraph 61, wherein the variant comprises amino acid substitutions V97I, Q246H, T127N, V171I, Q336E, D341N, G363A, and G379C.

Paragraph 65. The variant of paragraph 61, wherein the variant comprises amino acid substitutions V93D, V97I, Q246H, T127N, V171I, Q336E, D341N, G363A, and G379C.

Paragraph 66. The variant of any of paragraphs 1-65, which has an improved thermal activity.

Paragraph 67. The variant of any of paragraphs 1-65, which has an improved thermostability

Paragraph 68. The variant of any of paragraphs 1-65, which has an improved thermal activity and thermostability.

Paragraph 69. The variant of any of paragraphs 1-65, which has an improved specific activity.

Paragraph 70. A polynucleotide encoding the variant of any of paragraphs 1-65.

Paragraph 71. A nucleic acid construct comprising the polynucleotide of paragraph 70.

Paragraph 72. An expression vector comprising the polynucleotide of paragraph 70.

Paragraph 73. A host cell comprising the polynucleotide of paragraph 70.

Paragraph 74. A method of producing a GH Family 5 endoglucanase variant, comprising:
a. cultivating the host cell of paragraph 75 under conditions suitable for expression of the variant; and
b. recovering the variant.

Paragraph 75. A transgenic plant, plant part or plant cell transformed with the polynucleotide of paragraph 72.

Paragraph 76. A method for obtaining a GH Family 5 endoglucanase variant, comprising introducing into a parent GH Family 5 endoglucanase an amino acid substitution at one or more positions corresponding to positions 127, 171, 341, 336, 93, 97, 246, and 379 of SEQ ID NO: 2, wherein the variant has endoglucanase activity; and recovering the variant.

Paragraph 77. A method of reducing the viscosity in a mash comprising adding a GH Family 5 endoglucanase variant according to the invention to the mash.

The present invention is further described by the following examples that should not be construed as limiting the scope of the invention.

### EXAMPLES

Example 1 describes the strains and vectors used in subsequent examples. Examples 2-5 describe the generation of random mutagenesis libraries of T. reesei EGII. Examples 6 and 7 relate to the expression of GH Family 5 endoglucanase variants from microculture and the high-throughput screening to identify modified GH Family 5 endoglucanase variants with increased thermoactivity and/or thermostability. Example 8 relates to methods for determining the temperature profiles of thermoactive GH Family 5 endoglucanase variants.

### Example 1

### Strains and Vectors

Saccharomyces cerevisiae strain YNL219C BY4742 [11993] (MATα his3Δ1 leu2Δ0 lys2Δ0 ura3Δ0 Δalg9) was obtained from ATCC (cat. No. 4011993). Escherichia coli strain DH5α (F-φ80lacZΔM15 Δ(lacZYA-argF)U169 recA1 endA1 hsdR17(rk−, mk+) phoA supE44 thi-1 gyrA96 relA1 λ−) was obtained from Invitrogen (cat. No. 18265-017). The YEp352/PGKxylss-Cel5A and YEp352/PGKxylss-Cel5A^{G363A} vectors are described in U.S. Publication No. 2013/0095554.

### Example 2

### T. reesei EGII Random Mutagenesis Libraries

Four random mutagenesis libraries of T. reesei EGII were constructed as follows: PCR was performed for 20 amplification cycles using 75 ng, 25 ng, 1.0 ng, or 0.1 ng of YEp352/PGKxylss-Cel5A as template with primers XynSS and PGK-term. This vector fragment and each final amplicon were transformed simultaneously and cloned by in vivo recombination into yeast strain YNL219C BY4742 [11993] (Butler et al., 2003).

XylSS
(SEQ ID NO: 4)
5′ GAT CGT CGA CAT GGT CTC CTT CAC CTC CCT C
PGK-term
(SEQ ID NO: 5)
5′ GCA ACA CCT GGC AAT TCC TTA CC

### Example 3

### T. reesei EGII-G363A Random Mutagenesis Libraries

A random mutagenesis library of T. reesei EGII-G363 was constructed as follows: PCR was performed for 20 amplification cycles using 0.5 ng of YEp352/PGKxylss-Cel5A^{G363A} as template with primers XynSS and 3PGK-term. This vector fragment and each final amplicon were transformed simultaneously and cloned by in vivo recombination into yeast strain YNL219C BY4742 [11993] (Butler et al., 2003).

XylSS
(SEQ ID NO: 4)
5′ GAT CGT CGA CAT GGT CTC CTT CAC CTC CCT C
PGK-term
(SEQ ID NO: 5)
5′ GCA ACA CCT GGC AAT TCC TTA CC

### Example 4

### T. reesei EGII-T127N-V171I-G363A-G379C Random Mutagenesis Libraries

Three random mutagenesis libraries of T. reesei EGII-T127N-V171I-G363A-G379C were constructed as follows: PCR was performed for 20 amplification cycles using 20 fmol, 2 fmol, or 0.2 fmol of YEp352/PGKxylss-EG2^{T127N-V171I-G363A-G379C} as template with primers NM066 and PGK-term. This vector fragment and each final amplicon were transformed simultaneously and cloned by in vivo recombination into yeast strain YNL219C BY4742 [11993] (Butler et al., 2003).

NM066
(SEQ ID NO: 6)
5′ TAC TAT CAC CAC TTC GAC CCG GCC
PGK-term
(SEQ ID NO: 5)
5′ GCA ACA CCT GGC AAT TCC TTA CC

### Example 5

### Construction of GH Family 5 Endoglucanase Variants Comprising Four or More Amino Acid Substitutions

Two T. reesei EGII aggregates, TrEGII(T127N-V171I-G363A-G379C) and TrEGII(T127N-V171I-Q336E-D341N-G363A-G379C), were synthesized de novo by GenScript to include 5′-NheI and 3′-KpnI flanking restriction sites; these were received in the pUC57 vector. The modified Treg2 genes were excised using a NheI/KpnI double digest and ligated into a correspondingly linearized PGK-xyn2ss-6H vector.

Further modifications to TrEGII were introduced using a two-step PCR method involving megaprimer synthesis followed by megaprimer PCR (Table 2). The template for the first series of PCR reactions (PGK-xyn2ss-6H-TrEGII (V97I-T127N-V171I-Q336E-D341N-G363A-G379C)) was derived from a thermostable clone identified during the random mutagenesis screening of the error prone PCR libraries described in Example 4. The internal primers were modified to introduce the desired amino acid substitutions into the TrEGII (V97I-T127N-V171I-Q336E-D341N-G363A-G379C) construct. The external plasmid primers DK510 (SEQ ID NO: 7) and PGKterm (SEQ ID NO: 5) were used to amplify the final product. Megaprimers and final products were purified using the Wizard® SV Gel and PCR Clean-Up System.

TABLE 2
Generation of the modified TrEG2 enzymes by PCR.

| PCR | Step | Template | 5′ Primer | 3′ Primer | Amplicon |
|---|---|---|---|---|---|
| 1 | 1 | PGK-xyn2ss-6H- | DK510 | DK530 | PCR 1 Step 1 |
| | | TrEG2(V97I; T127N; V171I; | | | |
| | | Q336E; D341N; G363A; G379C) | | | |
| | 1 | PGK-xyn2ss-6H- | DK529 | PGKterm | PCR 1 Step 1 |
| | | TrEG2(V97I; T127N; V171I; | | | |
| | | Q336E; D341N; G363A; G379C) | | | |
| | 2 | PCR 1 Step 1 megaprimers | DK510 | PGKterm | 6H-TrEG2 (V97I; T127N; V171I; |
| | | | | | Q246H; Q336E; D341N; G363A; |
| | | | | | G379C) |
| 2 | 1 | 6H-TrEG2(V97I; T127N; V171I; | DK510 | DK528 | PCR 2 Step 1 |
| | | Q246H; Q336E; D341N; G363A; | | | |
| | | G379C) | | | |
| | 1 | 6H-TrEG2(V97I; T127N; V171I; | DK527 | PGKterm | PCR 2 Step 1 |
| | | Q246H; Q336E; D341N; G363A; | | | |
| | | G379C) | | | |
| | 2 | PCR 2 Step 1 megaprimers | DK510 | PGKterm | 6H-TrEG2 (V93D; V97I; T127N; |
| | | | | | V171I; Q246H; Q336E; D341N; |
| | | | | | G363A; G379C) |
| 3 | 1 | 6H-TrEG2(V97I; T127N; V171I; | DK510 | DK532 | PCR 3 Step 1 |
| | | Q246H; Q336E; D341N; G363A; | | | |
| | | G379C) | | | |
| | 1 | 6H-TrEG2(V97I; T127N; V171I; | DK531 | DK534 | PCR 3 Step 1 |
| | | Q246H; Q336E; D341N; G363A; | | | |
| | | G379C) | | | |
| | 1 | 6H-TrEG2(V97I; T127N; V171I; | DK533 | PGKterm | PCR 3 Step 1 |
| | | Q246H; Q336E; D341N; G363A; | | | |
| | | G379C) | | | |
| | 2 | PCR 3 Step 1 megaprimers | DK510 | PGKterm | 6H-TrEG2(V97I; T127N; V171I; |
| | | | | | Q246H; D294N; Q336E; D341N; |
| | | | | | Q344R; G363A; G379C) |
| 4 | 1 | 6H-TrEG2(V97I; T127N; V171I; | DK510 | DK528 | PCR 4 Step 1 |
| | | Q246H; D294N; Q336E; D341N; | | | |
| | | Q344R; G363A; G379C) | | | |
| | 1 | 6H-TrEG2(V97I; T127N; V171I; | DK527 | PGKterm | PCR 4 Step 1 |
| | | Q246H; D294N; Q336E; D341N; | | | |
| | | Q344R; G363A; G379C) | | | |
| | 2 | PCR 6 Step 1 megaprimers | DK510 | PGKterm | 6H-TrEG2(V93D; V97I; T127N; |
| | | | | | V171I; Q246H; D294N; Q336E; |
| | | | | | D341N; Q344R; G363A; G379C) |

The final PCR products were recombined (In-Fusion Recombinase—Clontech) into vector PGK-xyn2ss-6H linearized with NheI+KpnI. The recombinase mix was transformed into DH5α chemically-competent E. coli cells, plasmid extracted, and sequenced.

DK510
(SEQ ID NO: 7)
5′-TGGCTGTGGAGAAGCGC-3′
PGKterm
(SEQ ID NO: 5)
5′-GCAACACCTGGCAATTCCTTACC-3′
DK527
(SEQ ID NO: 8)
5′-GGCACTTGCGACACCTCGAAGATTTATCCTCC-3′
DK528
(SEQ ID NO: 9)
5′-CGAGGTGTCGCAAGTGCCATCTGTGGTAC-3′
DK529
(SEQ ID NO: 10)
5′-GCTACGTCGCACTTCATCTCTTTGCCTGGAAATG-3′
DK530
(SEQ ID NO: 11)
5′-GAGATGAAGTGCGACGTAGCACCAGC-3′
DK531
(SEQ ID NO: 12)
5′-CTTGGACTCAAACAACTCCGGTACTCACG-3′
DK532
(SEQ ID NO: 13)
5′-CCGGAGTTGTTTGAGTCCAAGTATTTGTGCAC-3′
DK533
(SEQ ID NO: 14)
5′-CATGTGCCGCCAAATCCAATATCTCAACCAGAA-3′
DK534
(SEQ ID NO: 15)
5′-ATTGGATTTGGCGGCACATGTTTTGTATGCAG-3′

### Example 6

### Expression and Isolation of GH Family 5 Endoglucanase Variants from Yeast Microplate Cultures

This example describes the selection and expression of TrEG2 variants from Saccharomyces cerevisiae for use in a high-throughput screening assay.

Saccharomyces cerevisiae transformants from the libraries described in Examples 2, 3 and 4 were grown on plates containing synthetic complete medium (SC: 2% agar w/v, 0.17% yeast nitrogen base w/v, 0.078%-Ura drop-out supplement w/v, 2% glucose w/v, 2% casamino acids w/v, 0.5% ammonium sulfate w/v, pH 5.5) and 0.12% Azo-CMC (Megazyme) for 3 days at 30° C.

Colonies showing visible clearing halos were selected for liquid media cultures by toothpick inoculation of 1 mL synthetic complete media (SC: 0.17% yeast nitrogen base w/v, 0.078%-Ura drop-out supplement w/v, 2% glucose w/v, 2% casamino acids w/v, 0.5% ammonium sulfate w/v, pH 5.5) in 96-deepwell microplates. Cultures were grown for 3 days at 30° C. and 250 rpm with humidity control. Glycerol stocks were made combining 100 μL of culture with 100 μL of 30% glycerol and stored at −80° C. Deepwell culture plates were then centrifuged at 3000 rpm for 5 minutes to pellet cells and supernatant was aspirated for screening assays.

### Example 7

### Screening of T. reesei EGII Libraries for GH Family 5 Endoglucanase Variants with Increased Thermoactivity

This example describes the screening of modified Trichoderma reesei TrEG2 cellulases for improved thermoactivity relative to the parental TrEG2 that had been cloned into Saccharomyces cerevisiae.

An aliquot of supernatant (50 μL) from each parental and variant microculture, produced as in Example 5, was added to 50 μL of 1.0% barley β-glucan (Megazyme; medium viscosity) buffered with 50 mM citrate phosphate at pH 5.0 and incubated at 70° C. for 1 hour. An identical assay was performed at 86° C. Microculture supernatants for the 70° C. assay were diluted 1 in 32 while supernatants for the 86° C. assay were diluted 1 in 8. Both assays were performed in a PCR plate and incubations were performed in a Tetrad thermalcycler. Contained in each 96-well PCR plate were six parental TrEG2 controls used for comparison. Assays were done in duplicate. Following each incubation, 80 μL of dinitrosalicyclic acid was added and the plates were heated to 95° C. for 5 min. A 135 μL aliquot of the solution was transferred to a microplate and the absorbance at 560 nm was measured.

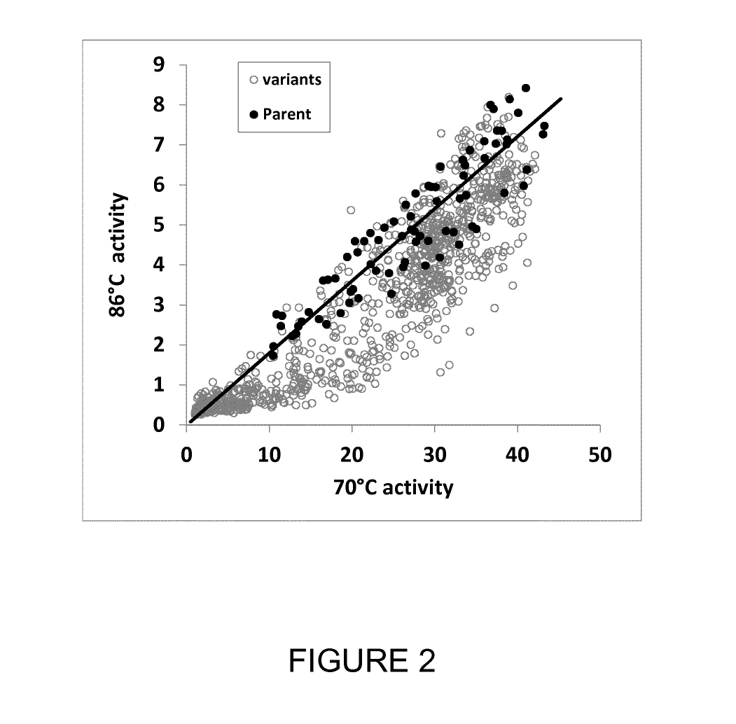

The absorbance at 70° C. and 86° C. was multiplied by 32 and 8, respectively (supernatant dilution factor) for all variants and parental controls. A high/low temperature activity ratio was then calculated by dividing the corrected absorbance at 70° C. by the corrected absorbance at 86° C. for each of the six parental controls and an average and standard deviation was calculated. A high/low temperature activity ratio was also calculated for each TrEG2 variant. Positives were then selected greater than 2 standard deviations above the average parental control. All positive modified TrEG2s were re-screened to reduce the number of false positives. Screening data from one complete round of screening can be found in FIG. 2.

### Example 8

### Determining the Temperature Profile for Selected GH Family 5 Endoglucanase Variants

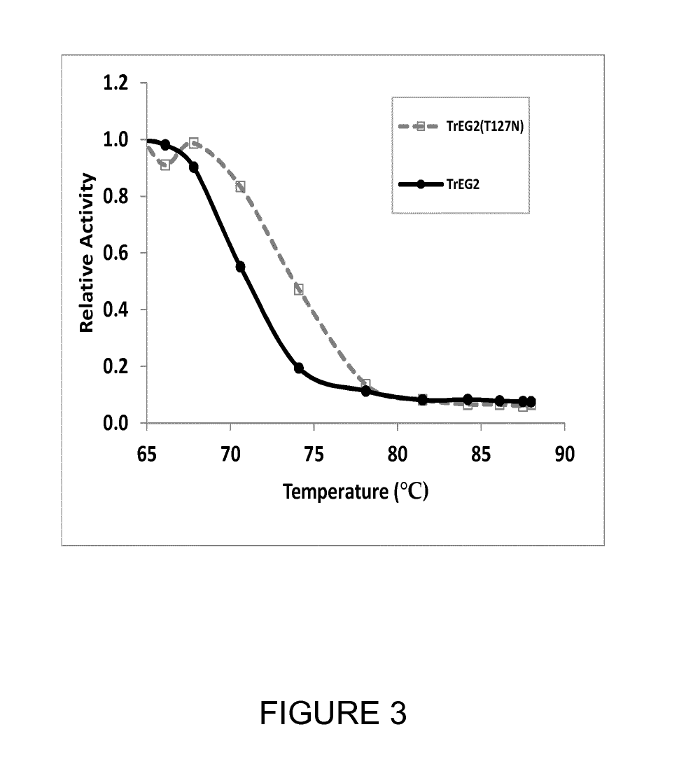

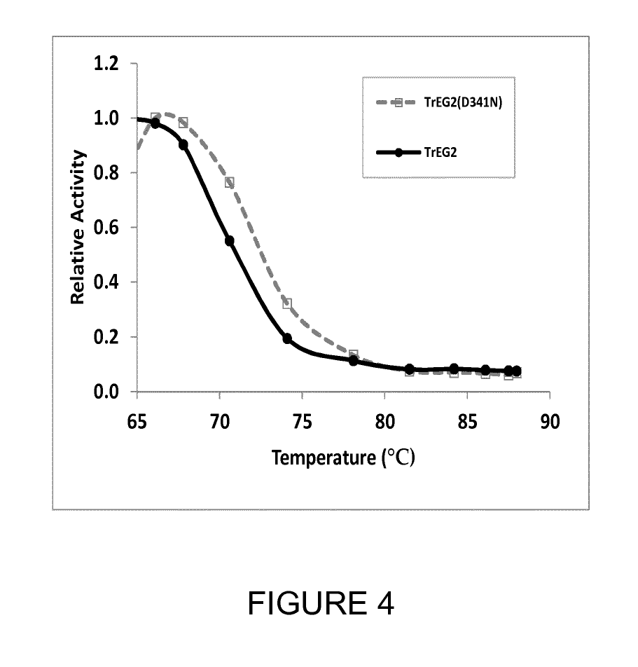

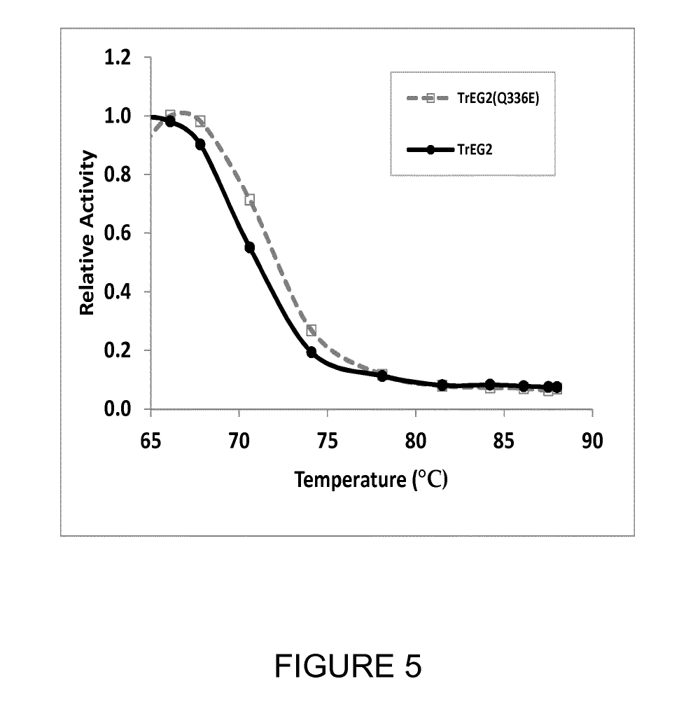

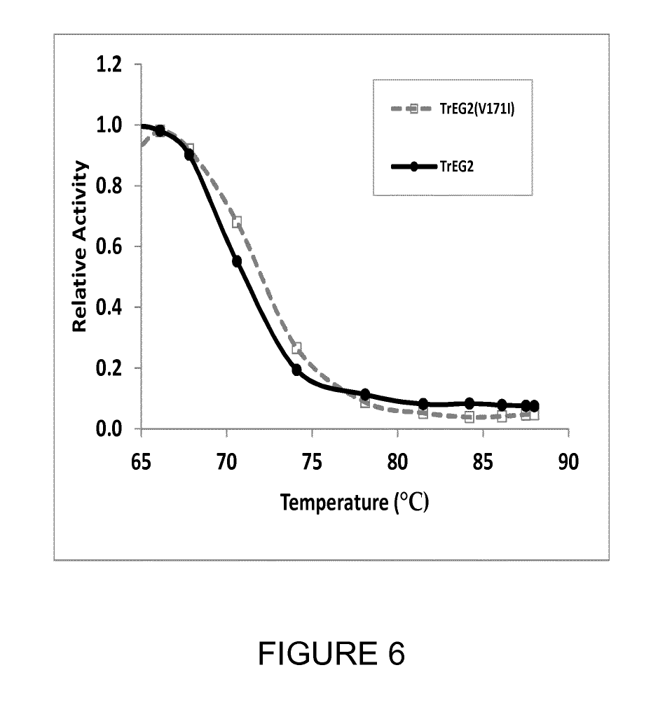

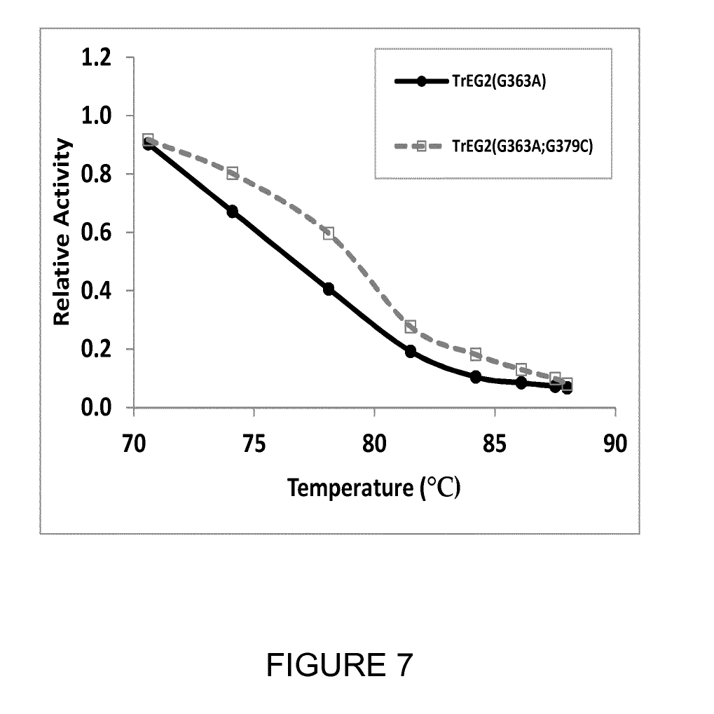

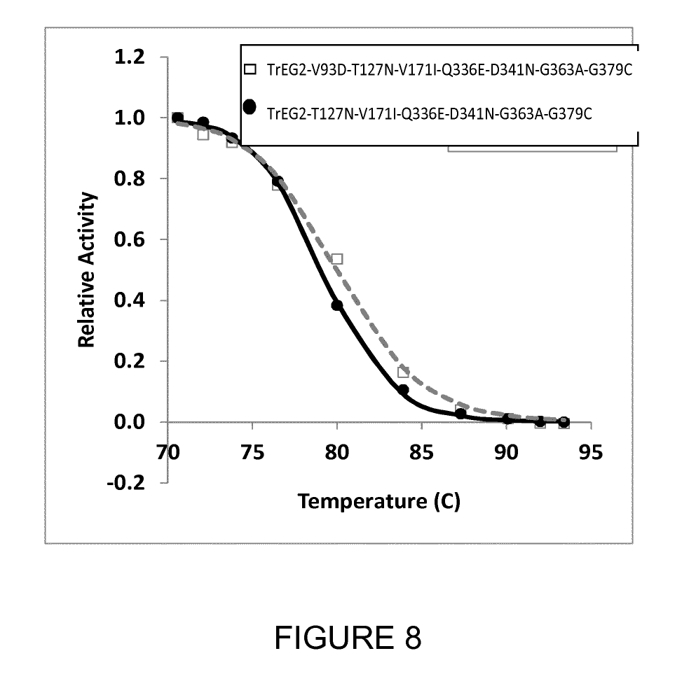

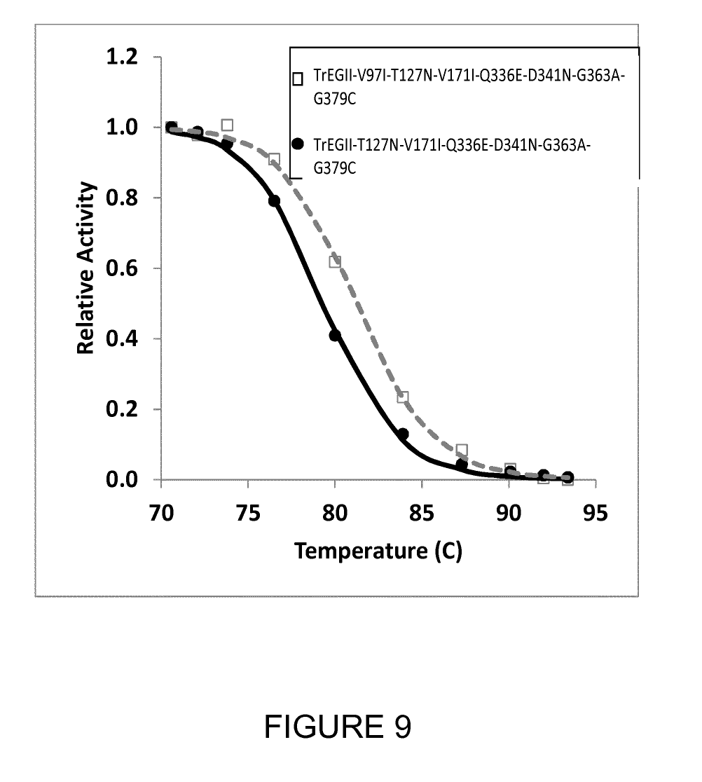

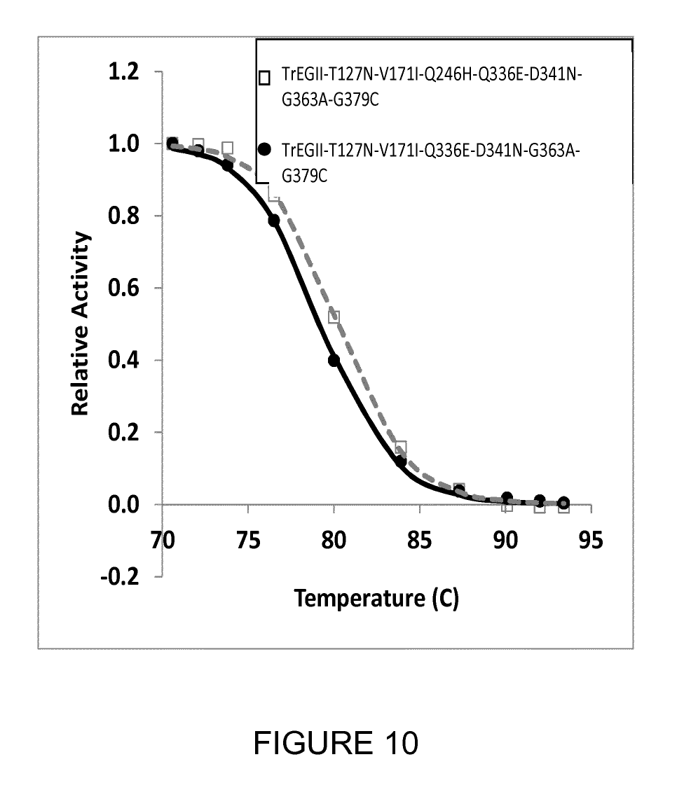

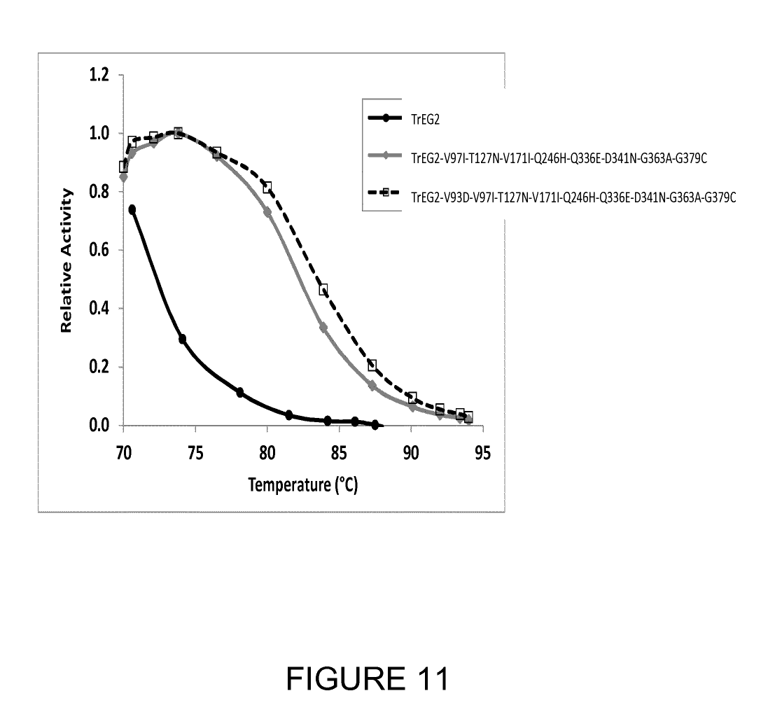

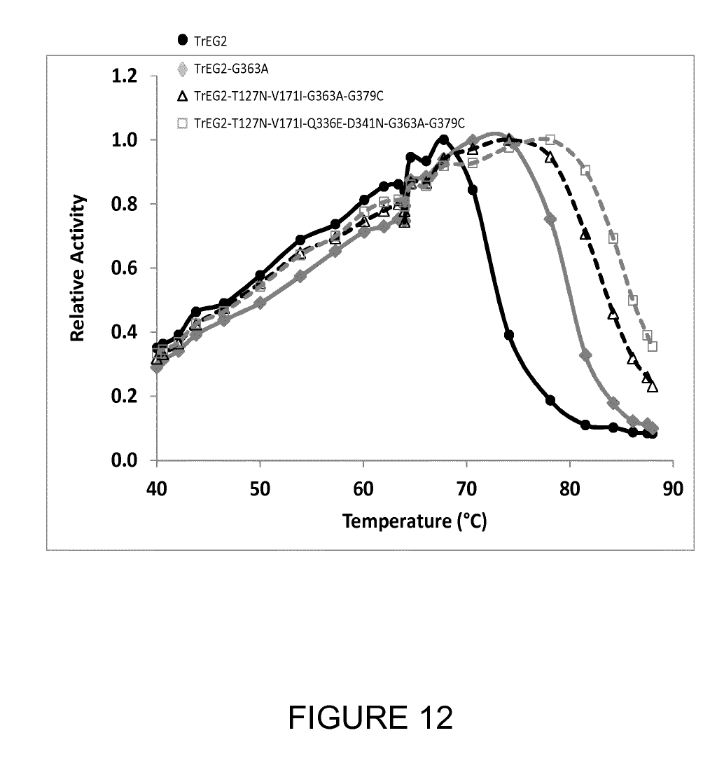

An aliquot of supernatant (50 μL; diluted 16-fold) from selected microcultures was added to 50 μL of 1.0% barley β-glucan (Megazyme; medium viscosity) buffered with 50 mM citrate phosphate at pH 5.0 and incubated at various temperatures for 1 hour. These assays were performed in a PCR plate and incubations were performed in a Tetrad thermalcycler. Assays were done in duplicate. Following each incubation 80 μL of dinitrosalicyclic acid was added and the plates were heated to 95° C. for 5 min. A 135 μL aliquot of the solution was transferred to a microplate and the absorbance at 560 nm was measured. Temperature profiles for selected TrEG2 variants and parental controls can be found in FIGS. 3-12.

1. A GH Family 5 endoglucanase variant, comprising an amino acid substitution at one or more positions corresponding to positions 127, 171, 341, 336, 93, 97, 246, and 379 of SEQ ID NO: 2, wherein the variant has endoglucanase activity, and wherein the variant has at least 80%, e.g., at least 85%, at least 90%, at least 95%, at least 96%, at least 97%, at least 98%, or at least 99% sequence identity to amino acids 71-397 of SEQ ID NO: 2.

2. The variant of claim 1, which is a variant of a parent GH Family 5 endoglucanase selected from the group consisting of:
a. a polypeptide having at least 80% sequence identity to amino acids 71-397 of SEQ ID NO: 2;
b. a polypeptide encoded by a polynucleotide that hybridizes under at least medium stringency conditions with (i) the mature polypeptide coding sequence of SEQ ID NO: 1, (ii) the cDNA sequence thereof, or (iii) the full-length complement of (i) or (ii);
c. a polypeptide encoded by a polynucleotide having at least 80% identity to the mature polypeptide coding sequence of SEQ ID NO: 1 or the cDNA sequence thereof; and
d. a fragment of SEQ ID NO: 2, which has endoglucanase activity.

3. The variant of any of claim 1, wherein the number of amino acid substitutions is 1-20, e.g., 1-10 or 1-5, such as 1, 2, 3, 4, 5, 6, 7, 8, 9, 10, 11, 12, 13, 14, 15, 16, 17, 18, 19, or 20, amino acid substitutions.

4. The variant of claim 1, which comprises one or more substitutions selected from the group consisting of T127N, V171I, D341N, Q336E, V93D, V97I, Q246H, and G379C.

5. The variant of claim 1, which further comprises an alteration at positions corresponding to position 363.

6. The variant according to claim 5, which further comprises G363A.

7. The variant of claim 1, which has an improved thermal activity, thermo stability, or both.

8. A polynucleotide encoding the variant of any of claim 1.

9. A nucleic acid construct comprising the polynucleotide of claim 8.

10. An expression vector comprising the polynucleotide of claim 8.

11. A host cell comprising the polynucleotide of claim 8.

12. A method of producing a GH Family 5 endoglucanase variant, comprising:
a. cultivating the host cell of claim 11 under conditions suitable for expression of the variant; and
b. recovering the variant.

13. A transgenic plant, plant part or plant cell transformed with the polynucleotide of claim 8.

14. A method for obtaining a GH Family 5 endoglucanase variant, comprising introducing into a parent GH Family 5 endoglucanase an amino acid substitution at one or more positions corresponding to positions 127, 171, 341, 336, 93, 97, 246, and 379 of the mature polypeptide of SEQ ID NO: 2, wherein the variant has endoglucanase activity; and recovering the variant.

15. A method of reducing the viscosity in a mash comprising adding a GH Family 5 endoglucanase variant according to claim 1 to the mash.

16. A method for degrading a cellulosic or hemicellulosic material, comprising treating the cellulosic or hemicellulosic material with an enzyme composition comprising a GH Family 5 endoglucanase variant according to claim 1.
